(19) United States
(12) Patent Application Publication
(10) Pub. No.: US 2017/0246111 A1
(43) Pub. Date: Aug. 31, 2017

(54) ACTIVE-LOADED PARTICULATE MATERIALS FOR TOPICAL ADMINISTRATION

(71) Applicant: PHARMASOL GMBH, Berlin (DE)

(72) Inventors: Frederik Hendrik Monsuur, Hasselt (BE); Hans Hermann Hoefer, Weshofen (DE); Cornelia Keck, Schwielowsee (DE)

(21) Appl. No.: 15/508,393

(22) PCT Filed: Sep. 15, 2015
(86) PCT No.: PCT/EP2015/071138

§ 371 Date: Mar. 2, 2017

Related U.S. Application Data
(60) Provisional application No. 62/050,587, filed on Sep. 15, 2014.
(60) Provisional application No. 62/144,555, filed on Apr. 8, 2015.
(60) Provisional application No. 62/212,425, filed on Aug. 31, 2015.

(51) Int. Cl. A61K 9/14; A61K 31/7048; A61K 38/13; A61K 47/44; A61K 47/32; A61K 31/7052; A61K 9/00

(52) U.S. Cl. CPC A61K 9/143; A61K 31/7052; A61K 9/146; A61K 31/7048; A61K 9/0014; A61K 47/44; A61K 47/32; A61K 38/13; A61K 9/0046; A61K 9/0017; A61K 9/006; A61K 9/0043; A61K 9/0048

(57) ABSTRACT

Compositions containing an amorphous biologically active ingredient and porous particles materials are disclosed. Methods of making and using the compositions to provide topical and/or dermal compositions for the treatment of humans and animals are also disclosed.

### FIELD OF THE INVENTION

The present invention relates to the field of drug delivery. In particularly, the present invention relates to compositions and methods of use thereof for the topical delivery of biological actives, e.g. cosmetic, cosmeceuticals and/or pharmaceutical actives, through the skin and/or mucus membranes in humans and animals.

### BACKGROUND OF THE INVENTION

Poorly soluble biological actives (e.g. drugs) represent a problem for topical delivery, i.e. penetration into e.g. the skin or mucosa, or permeation. Penetration into the skin is driven by the concentration gradient of the dissolved active in the formulation and the skin. However, the saturation solubility of poorly soluble drugs is very low, resulting in a very low concentration gradient.

For example water soluble vitamin C dissolved in the water phase of a dermal formulation can have a maximum concentration of about 0.3 g/ml, i.e. this is its saturation solubility (“Cs”) at 20° C. Immediately after application to the skin (time t=0), the vitamin C concentration in the skin (“Ct”) is zero, that means the concentration gradient Cs-Ct is 0.3 g/ml. In contrast, poorly soluble rutin has a solubility in water of about 0.13 mg/ml (=130 μg/ml) (Krewson, C. F.; Naghski, J. (2006). Some Physical Properties of Rutin. Journal of the American Pharmaceutical Association 41 (11): 582-7). The concentration gradient is almost a factor 3,000 lower, thus a priori the diffusive flux according to the 1st Fick law and the Noyes-Whitney equation about 3000 times lower. The solution of the state of the art to this problem was to increase the solubility of the active.

Where the active is oil soluble, this can be done very simply by using an oil-in-water cream (o/w cream) and dissolving the active, e.g. coenzyme Q10, in the oil phase of the cream. However, penetration depends not only on the concentration gradient, but also on the lipophilicity of the molecule and its partitioning (partition coefficient log P_{octanol/water}=log(concentration solute in octanol/concentration solute in water)). The lipophilic coenzyme Q10 likes rather to stay in the lipophilic environment of the oil droplets of the cream, than partitioning to the water phase and the skin (mixed hydrophilic-lipophilic environment). This fact is reflected in the old pharmaceutical rule, that penetration into the skin from suspension formulations is better than from formulation with dissolved active such as the oil-in-water creams. The active in suspension formulations is partially dispersed as particles (crystals) and partially dissolved but at a low concentration. The dissolved lipophilic molecules desire to leave the “uncomfortable environment” of the hydrophilic phase and partition into the relatively more lipophilic skin. Thus a suspension formulation—as the described formulations of this invention—is from the principle more desirable for topical delivery.

Analogous to oil droplets, poorly water-soluble but lipid-soluble molecules can be incorporated in lipidic particles or nanoparticles, for example liposomes, cubosomes, solid lipid nanoparticles (SLN) (Müller, R. H., Mäder, K., Gohla, S., Solid Lipid Nanoparticles (SLN) for Controlled Drug Delivery—A Review of the State of the Art, Eur. J. Pharm. Biopharm. 50, 161-177, 2000); and Müller, R. H., Radtke, M., Wissing, S. A., Solid Lipid Nanoparticles (SLN) and Nanostructured Lipid Carriers (NLC) in Cosmetic and Dermatological Preparations. ADDR Reviews 54 Suppl. 1, S131-S155, 2002). This increases the solubility in the formulation identical to o/w creams, but has the identical cream related problem described above. This problem can partially be compensated by the occlusion effect created by e.g. SLN and NLC, occlusion increases penetration of molecules.

Another approach to increase solubility is complexation which is often performed using cyclodextrins or polymers (e.g. polyethylene glycol). However this is not a universal solution as the molecule needs to fit from their size into the cyclodextrin (CD) ring or needs to be able to form a polymer complex. In addition, release from CD and polymer complexes can be very slow in case of high binding/association constants.

Since about one decade the “gold standard” to increase solubility is the concept of nanocrystals. Nanocrytals were introduced to the pharmaceutical market with the oral product Rapamune (Junghanns, J. U., Müller, R. H., Nanocrystal Technology, Drug Delivery and Clinical Applications. International Journal of Nanomedicine, Vol. 3, 2008) in the year 2000 and entered the cosmetic market in 2007 (=first topical products). Dermal products are e.g. in the line JUVEDICAL (Age-decoder Face Cream, Age-decoder Face Fluid) from the company Juvena Switzerland (rutin nanocrystals) and platinum rare from the company La Prairie, Switzerland (hesperidin nanocrystals). Nanocrystals are crystals in the nanodimension, i.e. a few nanometer to <1,000 nm (<1 μm). Due to the nano-dimension they have different physico-chemical properties compared to bulk material. Compared to bulk material they have an increased saturation solubility (Mauludin, R., Müller, R. H., Keck, C. M., Kinetic Solubility and Dissolution Velocity of Rutin Nanocrystals. Eur. J. of Pharm. Sciences 36, 502-510, 2009), thus increased concentration gradient to the skin and consequently an increased diffusive flux.

Mauludin reports a saturation solubility Cs of 124.4 μg/ml for rutin raw powder and 133.3 μg/ml as nanocrystals (Mauludin, R., PhD Thesis Nanosuspensions of Poorly Soluble Drugs for Oral Administration, Free University of Berlin, 2008, page 173), about 20 μg/ml for hesperidin raw powder, but about 80 μg/ml as nanocrystals, at 25° C. in water respectively (Mauludin, R., PhD Thesis Nanosuspensions of Poorly Soluble Drugs for Oral Administration. Free University of Berlin, 2008, page 176). Besides increased saturation solubility Cs, the nanocrystals have a higher dissolution rate dc/dt (Noyes-Whitney equation) compared to bulk material in the micrometer size range, which is due to the larger surface area and increased saturation solubility Cs.

The nanocrystals are dispersed in the water phase of a dermal formulation. The increased concentration gradient increases flux into the skin. Molecules penetrated from the dermal formulation into the skin are immediately replaced by molecules dissolving fast from the nanocrystals acting as depot in the dermal formulation. From the technical side, nanocrystals can be considered as presently optimal formulation approach for the dermal delivery of poorly soluble actives.

### STATE OF THE ART

It is known that amorphous materials have a higher saturation solubility than crystalline materials. Thus to increase the solubility, it is advantageous to use molecules in the amorphous state. Amorphous materials, however, have the tendency to re-crystallize. Re-crystallization is particularly favored when the amorphous material is in contact with liquid (water, oils, organic solvents), which leads to partial dissolution (until saturation solubility Cs of the amorphous material is reached). This initiates the re-crystallization process, e.g. as it occurs in Ostwald ripening. From these theoretical considerations, amorphous actives are only promising in dry oral formulations (e.g. tablets, powders in capsules), which would exclude the use of amorphous actives for dermal formulations.

Creation of amorphous state was realized by loading actives in the pores of porous material for oral administration (see, for example, PCT/EP2009/057688, WO2009/153346A2, US2012/0196873A1). It could be shown for the dry state, that loading resulted in actives which stayed amorphous for more than 3 years (Müller, R. H., Wei, Q., Keck, C. M., Stability of Industrially Feasible Amorphous Drug Formulations Generated in Porous Silica. W5313, AAPS Annual Meeting, San Antonio, 10-14 November 2013). The technology was suggested to have a performance in increasing oral bioavailability. No data about stability of the described amorphous state in liquid media was reported.

Porous materials were loaded with actives for application to the skin. However, no increase in penetration was reported compared to traditional dermal formulations (e.g. emulsions). Also no amorphous state was reported to be found in the porous materials when incorporated into dermal formulations.

Aminopropyl-functionalized mesoporous silica particles (MCM-41-mobile crystalline material) (Kresge, C. T.; Leonowicz, M. E.; Roth, W. J., Vartuli, J. C.; Beck, J. S. Nature. 1992, 359, 710) were used to study the dermal penetration of active in vitro (Mesoporous Silica as Topical Nanocarrier of Quercetin, S. Sapino, E. Ugazio, L. Gastaldi, G. Berlier, S. O. Bosso, D. Zonari, abstract booklet of conference by APGI (Association Pharmaceutique Galenique Industrielle), 3rd Conference Innovation in Drug Delivery—Advances in Local Drug Delivery. Sep. 22-25, 2013. Pisa, Italy—page 101).

The result of this poster presentation showed that in vitro penetration (amount retained in porcine skin after 24 hours) is not better from quercetin-loaded MCM-41 compared to hydroalcoholic solution of quercetin. Retained amount in porcine skin is even lower from MCM-41 in emulsion formulation compared to quercetin in emulsion. Quercetin loaded MCM-41 shows increased cell viability in cell culture compared to quercetin. However, similar increase in viability was observed from unloaded MCM-41. This just shows that the increase in viability was due to a lack of release of quercetion from the MCM-41. A non-releasing carrier is not suitable for dermal delivery. The conclusion as stated by the authors was that “. . . in vitro skin permeation profiles exhibited that silica nanoparticles did not significantly affect the skin uptake of quercetin”. This teaches away from using porous materials for topical delivery.

Mesoporous particles have also been used to load UV filters (see, for example, WO2009138513A2). Declared aims of the invention disclosed in this reference was (a) to avoid chemical degradation of UV filters by encapsulation, because degradation creates free radicals damaging the skin, and b) to retain the UV filter on the surface and not penetrate it into the skin, which is achieved by loading it into the pores. This teaching clearly teaches away from using porous particles as penetration enhancing system.

The sunscreen benzophenone-3 (BP-3) was incorporated into mesoporous silica for dermal application (see Mesoporous Silica Aerogel® as a Drug Carrier for the Enhancement of the Sunscreen Ability of Benzophenone-3. C. C. Li, Y. T. Chen, Y. T. Lin, S. F. Sie, Y. W. Chen-Yang, Colloids and Surfaces B: Biointerfaces 115 (2014) 191-196). Incorporation increased the sun protection efficiency of the BP-3 because it stayed fixed in the mesoporous carrier. The in vitro dissolution study showed a much slower dissolution of the BP-3 from the mesoporous silica (40.7-60.0%) compared to the compound BP-3 itself For dermal delivery one needs improved penetration promoted by fast release. However, such a retarded release tends to worsen the penetration conditions.

Polymeric particles known to localize in the hair follicles have been used to target the sebaceous glands via the hair follicles (see J. Lademann, H. Richter, et al. (2007). Nanoparticles—An Efficient Carrier for Drug Delivery into the Hair Follicles. Eur J Pharm Biopharm 66(2): 159-164). Also porous particles, being loaded with pharmaceutical actives, acted as the carrier to the follicles (see US Patent Publication No. 20120076841A1). These particles were used for administration of a cosmetically active compound into a pilosebaceous unit. The particles localize to a higher extent in the pilosebaceous unit via particle diffusion, massaging further enhancing localization, and release the active in the pilosebaceous unit. The particles diffuse into the gap around the hair shaft, hair root and hair bulb. For this localization only a particulate carrier is needed, e.g. also polymeric microspheres or liposomes can act as carrier. The reference shows that the porous particles—alternatively to microspheres and liposomes—are beneficial to localize active in the target site, i.e. the “pilosebaceous unit”. There is no teaching that active are loaded and maintained in the amorphous state, or that the particles have any benefits to deliver cosmetic or pharmaceutical active into the skin surface (epidermis) itself or mucus membranes, only to the skin appendage. Actives were loaded in to the particles by impregnation solutions of the desired active dissolved in a solvent, followed by precipitation of the solvent to provide the solid active. Normally precipitation from solvents leads to crystalline active.

Silicium based porous particles have also been used to incorporate optically active substances for application and action on the surface of the skin. Particles with diffusive reflection can improve the non-shininess of the finish, supported by optical substances incorporated into these particles (see US Patent Publication No. 20070183992A1). Optical brighteners were also incorporated into porous mineral particles (US Patent Publication No. 20050031559A1). Optically active substances and optical brighteners are substances which act on the surface of the skin, and which, due to toxicological reasons, should avoid or at least minimize being absorbed by the skin. From this it could be concluded, that one skilled in the art would not use particles which enhance undesired skin penetration. Rather, the porous particles should minimize absorption due to binding the substances into the pores. Consequently, using porous particle to promote skin uptake is in view of these publications against the state of the art.

Mesoporous material has also been used to change the appearance of biological surfaces such as the skin (see US Patent Publication No. 20080220026A1). In this reference, unloaded or loaded mesoporous particles were applied to enhance diffused transmittance of light, and giving a more aesthetic, smoother skin appearance. Additionally the mesoporous particles could be loaded with metal oxides (e.g. TiO₂, ZnO, Al₂O₃) or noble metal nanocrystals, and fluorescent materials, which is taught to further produce unique optical effects on skin. The aim of this reference was to improve the aesthetic or natural appearance of the skin by retaining the active materials on the skin surface by the encapsulation. There is no teaching regarding the use of the porous materials/particles for penetration enhancement.

Porous particles have frequently been used to incorporate liquids in their pores. The liquids can be hydrophilic or lipophilic, e.g. oils or fats. For example, moisturizing agents have been loaded onto spherical silica, e.g. aqueous solutions of proteins and amino acids, and polyhdydric alcohols (see U.S. Pat. No. 6,017,552). In US Patent Publication No. 20050220860A1, vitamin C is incorporated into a liposome of “liquid emulsion state” and then encapsulated, in addition jojoba oil was impregnated into the pores of porous powder of silica as second carrier, and both powders were blended. Encapsulation of the vitamin C-containing liposome increased the chemical stability of the active, and liposomes released on the skin promoted skin delivery via the liposomal effect. No penetration enhancing effects other than the liposomal effect were disclosed. Porous silica loaded with oil has been used in combination with a humectant in dermal formulations (see US Patent Publication No. 20050100565A9), again no penetration enhancement mechanism was described. Porous particles have also been used to absorb sebum from greasy skin to reduce the greasiness of the skin (see US Patent Publication No. 20060039938A1).

A composition with porous silicon structure has also been described for use on the human face (see US Patent Publication No. 20110229540A1). The compositions are suitable for “effective and controlled delivery of active ingredients.” The porous silicon containing composition are reported to be useful for targeted delivery of ingredients; extended release of ingredients; retention of significant levels of active ingredients on the face over extended periods of time, excellent skin feel and visual appearance. However, there is no evidence or data given in examples to support these benefits. In fact, the retention of significant levels of active ingredients on the face over extended periods of time diverts or teaches away from a bioavailability enhancement in the skin. When the active has an increased retention time on the skin, it penetrates less into the skin. Extended release often reduces skin penetration (less released active available for penetration).

Active-loaded porous silica particles have also been used for the prolonged release of actives in treating mucous membrane disease see WO2013098675 A1). Again prolonged release contradicts the penetration enhancement, special physical status of the loaded active and its positive effects on bioavailability are not reported. On the contrary, bioavailability enhancement is only described to be potentially achieved by combining the particles with additional principles, e.g. bioadhesion.

A topical composition comprising porous spherical disintegrative silica impregnated with water-insoluble skin benefit agents was described in US Patent Publication No. 20050074474A1. The particles were described to be disintegrative, that means they “are readily disintegrated upon spreading on the skin”, thus releasing the compound. As stated in US Patent Publication No. 20050074474A1, “water-insoluble skin benefit agents tend to provide unfavorable skin feel, and/or interfere with desirable product physical properties of the product. Any of such causes may result in a poor performing, or even unstable product.” The publication states that encapsulating the agent into particles can protect the ingredient from interacting with the product, but “the incorporated agent may not be fully utilized on the skin”, i.e. the bioavailability goes down. Thus disintegrative silica was used in US Patent Publication No. 20050074474A1. Due to the applied shear, the particles disintegrate and the water-insoluble agent becomes available directly to the skin. Upon disintegration, the released agent behaves as a “normally” incorporated agent in a dermal formulation, no bioavailability enhancement occurs, and consequently no bioavailability enhancement is described in US Patent Publication No. 20050074474A1.

Substance-supporting porous silica has also been described, (see US Patent Publication No. 20070003492A1). The porous particles were only substance-supporting particles. They were loaded with e.g. menthol as flavor or antibacterial polyphenols and incorporated into chewing gum, to achieve a prolonged release. There is no teaching about dermal use, the prolonged release teaches away from use as penetration enhancing delivery system on the skin. For penetration enhancement fast release creating a concentration as high as possible, and thus a concentration gradient as high as possible is desired.

Porous silicon-containing carriers loaded with an active ingredient hardly soluble in water were also used for the preparation of solid dispersions to be used in oral pharmaceutical compositions (e.g., tablets, granules, or capsules) (see U.S. Pat. No. 8,722,094 B2). Solid dispersions were known to increase the dissolution rate of drugs, and to increase bioavailability in case the bioavailability is dissolution velocity limited. For preparation of the solid dispersion drug, surfactant and polymer were dissolved in an organic solvent, the porous particles added and the solvent evaporated, to obtain a solid dispersion. Alternatively instead creating a solid dispersion, porous particles can be loaded with drug, e.g. using the impregnation method, and the obtained drug-loaded powder can be processed to a tablet or filled into a capsule (WO2009/153346A2). However neither discloses potential use in dermal formulations.

In conclusion, it can be summarized that unloaded and loaded porous particles were applied to the skin. Unloaded particles were used with the intention to remove material from the skin (e.g. absorbing sebum in greasy skin). Loaded particles were mainly used to create an effect on the skin (e.g. optical active substances, metal oxides for improving skin aspect), and were not generally intended to lead to a penetration of the loaded compounds. Such penetration was even undesired, thereby leading to encapsulating these substances in porous materials. From these porous materials retarded/prolonged release was reported. In case a loading was performed (e.g. quercetin) to investigate if better penetration could be achieved, no improvement in penetration compared to traditional formulations were reported. From this, loaded porous materials appeared not suitable to increase dermal drug delivery.

Consequently, there exists a need for topical and dermal delivery formulations having improved delivery of biological actives through the skin and/or mucous penetration into the human body, which compositions offer the advantages associated with or greater than compositions containing nanocrystal actives.

### SUMMARY OF THE INVENTION

We have now discovered compositions which are useful for topical delivery of biological actives which minimize problems heretofore associated with prior topical delivery compositions for poorly soluble biological actives. In accordance with the present invention, it was found that certain porous materials loaded with biological actives, e.g. cosmetic, cosmeceutical or pharmaceutical actives, in the amorphous state or partially amorphous state, i.e. having a crystallinity of less than 50% as determined by x-ray diffraction or differential scanning calorimetry (DSC), unexpectedly provide increased stability and performance for topical delivery of actives into the skin and mucosa as compared to similar dermal formulations comprising active nanocrystal compositions.

The porous compositions of the invention maybe incorporated into liquid media to provide topical formulations having improved dermal delivery of poorly soluble actives. When applied to the surface of the skin and/or mucus membrane, the dermal formulations show superior performance compared to the present standard to increase topical penetration, e.g. higher saturation solubility, higher suspension stability and superior penetration. Advantageously, the porous particles of the invention provide increased stability of the amorphous state in a liquid media environment and therefore provide increased stability in final dermal or topical formulations. Other advantages of the loaded porous particles of the invention include, but are not limited to, pleasing texture without sandy feeling, ease of production of the active loaded particles, ease of incorporation of the loaded particles into dermal formulations as well as more cost effective production as compared to the actives in the form of nanocrystals.

In one embodiment of the invention, the present invention comprises compositions comprising porous particles loaded with a biological in an amorphous state.

In another embodiment, the present invention comprises compositions comprising porous particles loaded with a biological in a partially amorphous state.

In yet another embodiment, the present invention comprises compositions comprising porous particles loaded with a biological active having a crystallinity of less than 50%, or less than 40%, or less than 30% or less than 20%, as determined by x-ray diffraction.

In one embodiment of the invention, the present invention comprises compositions comprising porous particles loaded with a biological active in a substantially amorphous state, wherein the inorganic oxide particles possess (a) pores having a pore volume of about 0.1 cm³/g or greater; (b) a average pore size of greater than or equal to about 2 nm and a surface area from about 10 m²/g to about 1000 m²/g, as measured by BET-Nitrogen absorption method.

In another embodiment, the present invention comprises compositions comprising porous particles loaded with a biological active in a partially amorphous state, wherein the inorganic oxide particles possess (a) pores having a pore volume of about 0.1 cm³/g or greater; (b) a median pore size of about 2 nm to about 30 nm and a surface area from about 10 m²/g to about 1000 m²/g, as measured by BET-Nitrogen absorption method.

In yet another embodiment, the present invention comprises compositions comprising porous particles loaded with a biological active having a crystallinity of less than 50%, or less than 40%, or less than 30% or less than 20%, as determined by x-ray diffraction or by differential scanning calorimetry (DSC), wherein the inorganic oxide particles possess (a) pores having a pore volume of about 0.1 cm³/g or greater; (b) a median pore size of about 2 nm to about 30 rim and a surface area from about 10 m²/g to about 1000 m²/g, as measured by BET-Nitrogen absorption method.

In a further embodiment, the present invention comprises compositions in accordance with of any of the above embodiments wherein the porous particles have an average diameter of from about 0.1 μm to about 1,000 μm.

In an even further embodiment, the present invention comprises compositions in accordance with of any of the above embodiments wherein the porous particles have an average diameter of less than 125 μm.

In yet a further embodiment, the present invention comprises compositions in accordance with of any of the above embodiments wherein the porous particles have an average diameter from greater than 125 μm to about 1000 μm.

In one embodiment, the present invention comprises compositions in accordance with any of the above embodiment wherein the porous particles are porous inorganic particles. In another embodiment, the present invention comprises compositions in accordance with any of the above embodiment wherein the porous particles are porous inorganic oxide particles.

In yet another embodiment, the present invention comprises compositions in accordance with any of the above embodiment wherein the porous particles are porous organic particles.

In one embodiment, the present invention provides topical and dermal formulations comprising the composition of any of the above embodiments which are dispersed in liquid media.

In another embodiment, the present invention provides topical and dermal formulations comprising the composition of any of the above embodiments having enhanced penetration of the actives into the skin and/or mucosa in humans and animals.

In another embodiment, the present invention provides topical or dermal formulations comprising compositions of any of the above embodiments having enhanced stability and performance of actives for delivery into the skin and/or mucosa membrane.

The present invention is further directed to methods of making the compositions of any of the above embodiments. In one embodiment, the method of making compositions in accordance with any of the above embodiment comprises incorporating at least one biological active into the pores of and/or on the surface of a porous inorganic oxide material in a manner such that the active is substantially or partially in an amorphous state. In another embodiment, the method of making compositions in accordance with any of the above embodiment comprises incorporating at least one biological active into the pores of and/or on the surface of a porous inorganic oxide material in a manner such that the active has a crystallinity of less than 50%, or less than 40%, or less than 30%, or less than 20%, as determined by x-ray diffraction or differential scanning calorimetry (DSC).

The present invention is further directed to methods of using the compositions of any of the above embodiments. In one embodiment, the method comprises incorporating the compositions of the invention into dermal or topical formulations such as gels, creams, e.g. oil-in-water creams, pastes, serums, lotions, oils, milks, sticks, ointments, solutions, suspensions, dispersions, or emulsions, and sprays, in a biologically active dosage. In another embodiment, the method of using comprises administering said dermal or topical formulation to the skin or mucosa of humans or animal so as to deliver a biological active through the skin and/or muscoa.

These and other features and advantages of the present invention will become apparent after a review of the following detailed description of the disclosed embodiments and the appended claims.

### BRIEF DESCRIPTION OF THE FIGURES

FIG. 1 depicts the XRD patterns for crystalline azithromycin raw drug powder and that of pure amorphous Syloid® SP53D-11920 silica.

FIG. 2 is a graphic representation of a DSC measurement of a physical mixture of salicylic acid and Syloid® SP53D-11920 silica at a ratio of 1:10.

FIG. 3 is a graphic representation of a DSC measurement of a porous material sample loaded with a large amount of salicylic acid solution (salicylic acid: Syloid® SP53D-11920 silica, 1:10 ratio).

FIG. 4 is a graphic representation of the dissolution profile of 32.0% loading Syloid® SP53D-11920 silica, 35.0% azithromycin physical mixture and coarse drug powder at 25° C. in Milli-Q® water.

FIG. 5 is a graphic representation of the saturation solubility of 32.0% azithromycin-loaded Syloid® SP53D-11920 silica after 40 min, and of azithromycin nanocrystals and raw drug powder in water after 60 minutes.

FIG. 6 is a graphic representation of the saturation solubility of 26.4% azithromycin-loaded Neusilin® US2 silica and azithromycin raw drug powder in water after 4 h shaking.

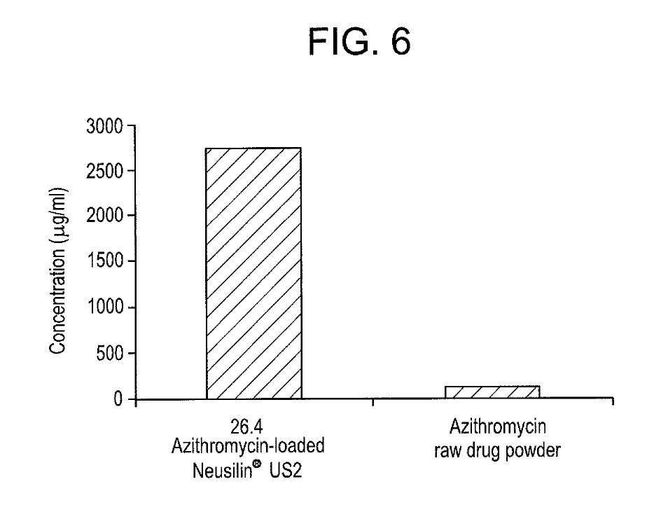

FIG. 7 depicts an X-ray diffraction patterns of Syloid® SP53D-11920 silica loaded with azithromycin loaded 32.0% on day 7 and day 30 showing the preservation of the amorphous state.

FIG. 8 depicts the results of a pig ear study plotted as the amount of azithromycin versus the number of tapes stripped from the skin.

FIG. 9 represents light microscopy pictures (160× fold magnification) of 5% unloaded Syloid® SP53D-11920 silica dispersed in water (left) and in a 5% hydroxpropylcellulose (HPC) gel after 4 months of storage, and azithromycin-loaded Syloid® SP53D-11920 silica in a 5% HPC gel (right) after 2 months of storage (all at room temperature).

FIG. 10 represents light microscopy pictures (160× fold magnification) of 5% unloaded Neusilin® US2 silica dispersed in water (left) and in a 5% HPC gel (right) after 4 months of storage at room temperature.

FIG. 11 represents light microscopy pictures (160× fold magnification) of 5% unloaded Aeroperl® 300 silica dispersed in water (left) and in a 5% HPC gel (right) after 4 months of storage at room temperature.

FIG. 12 is a graphic representation the loading of hesperidin onto Aeroperl® 300 silica: x-ray diffractogram of hesperidin (upper), physical mixture of hesperidin and Aeroperl® silica (middle) and 54% hesperidin loaded onto Aeroperl® silica (lower).

FIG. 13 is a graphic representation of the saturation solubilties (μg/ml) of amorphous hesperidin loaded onto Aeroperl® 300 silica, hesperidin nanocrystals and hesperidin raw powder as a function of time from 0.5 to 48 hours in different media.

FIG. 14 is a graphic representation of a pig ear skin study with rutin—Tape stripping (μg/strip) versus number of strips (19) after application of 5% rutin nanocrystal gel versus 1% rutin loaded onto porous silica Syloid® SP53D-11920 silica (n=2).

FIG. 15 is a graphic representation of a pig ear skin study with rutin—normalized plot by dividing amount (μg) per tape by the rutin concentration (%) in the applied formulation, i.e. plot of μg/% versus tape number (n=2). Formulations: 5% rutin nanocrystals (NC) in gel, and 1% rutin in Syloid® gel formulation.

FIG. 16 is a graphic representation of a pig ear skin study with hesperidin—Tape stripping (μg/strip) versus number of tape strips (30) after application of 5% hesperidin raw drug powder (RDP) in gel, 5% hesperidin nanocrystal gel versus 1% rutin loaded onto porous Syloid® SP53D-11920 silica (n=3).

FIG. 17 is a graphic representation of a pig ear skin study with hesperidin—normalized plot by dividing amount (μg) per tape by the hesperidin concentration (%) in the applied formulation, i.e. plot of μg/% versus tape number (n=3). Formulations: 5% hesperidin raw drug powder (RDP) in gel, 5% hesperedin nanocrystals (NC) in gel, and 1% hesperidin in Syloid® gel formulation.

FIG. 18 is a graphic representation of the x-ray diffractograms of cyclosporine raw drug powder (RDP) (upper) and of cyclosporine loaded into Syloid® SP53D-11920 silica showing the amorphous state in both formulations.

FIG. 19 is a graphic representation of the results of a pig ear skin study with cyclosporine—Tape stripping (μg/strip) versus number of strips (19) after application of 5% amorphous cyclosporine raw drug powder (RDP) gel versus 1% cyclosporine loaded onto porous Syloid® SP53D-11920 silica (n-3).

FIG. 20 is a graphic representation of the results of a pig ear skin study with cyclosporine—normalized plot by dividing amount (μg) per tape by the cyclosporine concentration (%) in the formulations: 5% amorphous cyclosporine raw drug powder (RDP) in gel, and 1% cyclosporine onto porous Syloid® SP53D-11920 silica in gel (n=3).

### DETAILED DESCRIPTION OF THE INVENTION

To promote an understanding of the principles of the present invention, descriptions of specific embodiments of the invention follow and specific language is used to describe the specific embodiment. It will nevertheless be understood that no limitation of the scope of the invention is intended by the use of specific language. Alterations, further modifications, and such further applications of the principles of the present invention discussed are contemplated as would normally occur to one ordinarily skilled in the art to which the invention pertains.

It must be noted that as used herein and in the appended claims, the singular forms “a”, “and”, and “the” include plural referents unless the context clearly dictates otherwise. Thus, for example, reference to “an oxide” includes a plurality of such oxides and reference to “oxide” includes reference to one or more oxides and equivalents thereof known to those skilled in the art, and so forth.

“About” modifying, for example, the quantity of an ingredient in a composition, concentrations, volumes, process temperatures, process times, recoveries or yields, flow rates, and like values, and ranges thereof, employed in describing the embodiments of the disclosure, refers to variation in the numerical quantity that may occur, for example, through typical measuring and handling procedures; through inadvertent error in these procedures; through differences in the ingredients used to carry out the methods; and like proximate considerations. The term “about” also encompasses amounts that differ due to aging of a formulation with a particular initial concentration or mixture, and amounts that differ due to mixing or processing a formulation with a particular initial concentration or mixture. Whether modified by the term “about” the claims appended hereto include equivalents to these quantities.

As used herein, the term “biological active/s” is used herein to mean compounds or molecules which generate biological activity in the body, including, but not limited to, cosmetic, cosmeceutical, pharmaceutical, medicinal or biological activity.

As used herein, the term “amorphous state” is used to mean that no crystalline fraction can be detected by X-ray diffraction.

As used herein, the term “dermal” is used relating to the skin surface and/or inside the skin layers.

As used herein, the term “inorganic oxides” mean binary oxygen compounds where the inorganic component is the cation and oxide is the anion. The inorganic material includes metals and may also include metalloids. Metals include those elements on the left of the diagonal line drawn from boron to polonium on the periodic table. Metalloids or semi-metals include those elements that are on the right of this line. Examples of inorganic oxide include silica, alumina, titania, zirconia, etc., and mixtures thereof.

As used herein, the term “liquid media” means media which are fluid with low viscosity to very high viscosity.

As used herein, the term “loaded” refers to porous particles, mean particles which contain actives in the pores or on the surface thereof, or simultaneously in the pores and on the surface thereof, as distinguished from the particulate material without the presence of any active.

As used herein, the term “non-ordered porous material” refers to porous particles possessing an internal structure such that they do not have a low angle X-ray diffraction pattern according to Bragg's Law. Such materials may be formed via any known process including, but not limited to, a solution polymerization process such as for forming colloidal particles, a continuous flame hydrolysis technique such as for forming fused particles, a gel technique such as for forming gelled particles, and a precipitation technique such as for forming precipitated particles. The particles may be subsequently modified by autoclaving, flash drying, super critical fluid extracting, etching, or like processes. The particles may be composed of organic and/or inorganic materials and combinations thereof. In one exemplary embodiment the particles are composed of inorganic materials such as inorganic oxides, sulfides, hydroxides, carbonates, silicates, phosphates, etc, but are preferably inorganic oxides. The particles may be a variety of different symmetrical, asymmetrical or irregular shapes, including chain, rod or lath shape. The particles may have different structures including amorphous or crystalline, etc. The particles may include mixtures of particles comprising different compositions, sizes, shapes or physical structures, or that may be the same except for different surface treatments. Porosity of the particles may be intra-particle or inter-particle in cases where smaller particles are agglomerated to form larger particles. In one exemplary embodiment the particles are composed of inorganic materials such as inorganic oxides, sulfides, hydroxides, carbonates, silicates, phosphates, etc, but are preferably inorganic oxides. Porous materials include organic and inorganic materials, or hybrids thereof, and may be in the form of particles, monoliths, membranes, coatings, and the like.

As used herein, the term “average pore diameter” as it refers to porous, particulate materials or particles, refers to the pore diameter below which 50% of the intra-particle pore volume resides. As used herein the term “pore size distribution” is used herein to mean the relative abundance of each pore size in a representative volume of porous inorganic particles.

As used herein, the terms “mucus membrane” and/or “muscoa” are used herein interchangeably to refer to linings in the body, both human and animal, of mostly endodermal origin, covered in epithelium, which are involved in absorption and secretion. They line cavities that are exposed to the external environment and internal organs. They are at several places contiguous with skin: at the nostrils, the lips of the mouth, the oral cavity, the eye and eyelids, the ears, the genital area, the anus, etc.

As used herein, the term “ordered porous material” refers to porous particles that have an internal structural order such that they possess a low angle X-ray diffraction patterns according to Bragg's Law. Such materials include ordered mesoporous silica, for example, MCM-41, SBA-15, TUD-1, HMM-33 and FSM-16.

As used herein, the term “partially loaded” refers to particles in which only a portion of the pores and/or surface of the particles are loaded with active/s.

As used herein, the term “pore volume” refers a pore volume as defined in C. H. Bartholomew, R. J. Farrauto: Fundamentals of Industrial Catalytic Processes. p. 80-84, John Wiley & Sons, 2006.

As used herein, the terms “porous particles” and “porous particulate materials” are used herein interchangeably to refer to particles having a structure containing pores; in particular, particles having a porous structure which permits the incorporation, at least in part, of one or more actives into the particles.

As used herein, the term “poorly soluble” refers to compounds requiring 100 to 1,000 ml of solvent for dissolution of 1 g compound. In case of water this corresponds to a dissolved concentration of 10mg/ml to 1 mg/ml. Very poorly soluble compounds are defined requiring between 1,000 ml to 10,000 ml solvent to dissolve 1 g compound, corresponding to a concentration range 1 mg/ml to 0.1 mg/. Highly poorly soluble compounds require more than 10,000 ml per gram, they dissolve in a concentration less than 0.1 mg/ml, i.e. less than 100 μg/ml. The present invention covers poor solubilities of 10 mg/ml or less, especially less than 1 mg/ml, and preferentially around and less than 0.1 mg/ml (=100 μg/ml).

As used herein, the term “skin” is refers to the outer covering of the body, human or animal.

As used herein, the term “substantially amorphous” is used herein to indicate a measurable crystallinity of 10% or less, preferably 5% or less, as determined by x-ray diffraction or DSC.

As used herein, the term “topical” is used herein to refer to the application to a surface of the body or in the body, being accessible from the outside, for example, but not limited to, to the skin, ocular mucosa, vaginal and rectal mucosa, mucosa of the lung surface or other mucus membranes of the body.

In accordance with the present invention, particulate materials particles are loaded with a biological active, such as for example, cosmetic, cosmeceutical or pharmaceutical active, in an amorphous state. In one embodiment of the invention the particulate materials are porous particles. In another embodiment of the invention, the particles are non-porous particles.

In one embodiment of the invention, the active is in a substantially amorphous state. In another embodiment of the invention the active is in a partially amorphous state such that only a portion of the active is in an amorphous state. In yet another embodiment of the invention, the active has a crystallinity of less than 50%, or less than 40%, or less than 30% or less than 20%, as determined by x-ray diffraction or differential scanning calorimetry (DSC). In another embodiment of the invention, the active has a crystallinity of about 50% to about 5%, as determined by x-ray diffraction.

Actives in the substantially or partially amorphous form are loaded into the pores and/or on the surface of the porous particulate materials, e.g. porous inorganic oxide materials such as, for example, Syloid° silica, Aeroperl® silica, Neusilin® silica (examples 1 to 3). The crystalline state of the actives may be determined by xray diffraction or differential scanning calorimetry (DSC) and should be at least partially amorphous for all loadings. Biological actives (e.g. cosmetic active, pharmaceutical drug etc.) may be loaded in the pores and partially on the surface of the porous particles. In one embodiment, the actives are located predominately in the pores of the porous particles. In another embodiment, when the loading is being performed using low concentration solutions (e.g. 10% active in ethanol well below saturation solubility), the actives may be located into the pores and/or on the surface of the particles. The higher amount of actives on the particles surface may be found when loading the porous materials of the invention with higher concentrated solutions of active (e.g. 30% active in ethanol closer to saturation solubility).

The amount of actives to be loaded into the pores and/or onto the particle surface depends on the desired biological effect. Actives may be present in the pores and/or on the surface of the porous particles in an amount ranging from about 0.0001% to about 95% by weight of the particles. In one embodiment the amount of active ranges from about 0.01 to about 70% by weight, and in particular, from about 1.0 to about 50 by weight, relative to the total weight of the particles once loaded.

Maximum loading capacity in the amorphous state may be determined by analyzing the porous particles with increasing drug load. The saturation solubility of the active may be determined by performing a dissolution experiment, i.e. adding excess compound to the solvent (e.g., water) and shaking it for hours or days until a plateau of solubility has been reached. For example, the maximum amount of azithromycin and Syloid® silica without detecting first peaks of crystalline material was 32.0% (example 4). The saturation solubility increase of azithromycin was determined comparing raw drug powder to azithromycin-loaded Syloid® silica and as control the physical mixture of azithromycin and Syloid® silica. The 32.0% azithromycin loaded Syloid® SP53D-11920 silica had an about 6 times higher saturation solubility in water (1300 μg/mL) compared to the physical mixture (213 μg/mL) and 14 times higher than that of raw drug powder (93 μg/mL) at 40 minutes (example 5).

Surprisingly it was found that the saturation solubility achieved with amorphous, i.e. substantially or partially amorphous, active loaded in the pores and/or on the surface of the porous materials was clearly superior to the Cs obtained with nanocrystals. The porous amorphous active-loaded material exhibited superior topical delivery based on the increase of the concentration gradient Cs-Ct.

Active material used in the compositions of the present invention may comprise any known cosmetic or biological active capable of forming and maintaining an amorphous state. The biological active used in the compositions of the present invention may comprise any known biologically active material. The term “biologically active ingredient” is meant to cover any pharmaceutical or other active ingredient for administration to humans or animals, in particular to warm-blooded animals. The biologically active material may be an active pharmaceutical ingredient, which comprises include natural, semi-synthetic or synthetic molecules. In some embodiments, the biologically active material comprises two or more active pharmaceutical ingredients in combination with one another. Other biologically active ingredients include ingredients that have an effect on the general well-being or have an effect on the outer appearance (cosmetic or cosmeceutical) such as the skin, hair, lips, and eyes. Such ingredients include any agents for use in cleansing, beautifying, promoting attractiveness, or altering the appearance, for example moisturizers, oils, anti-wrinkle agents, fragrances, and the like. Also included are ingredients for nutritious applications (in particular the so-called “nutraceutical” ingredients). Such ingredients include food supplements such as, for example, dietary food supplements, vitamins, minerals, fiber, fatty acids, and amino acids. Examples of such ingredients are vitamin C, omega-3 fatty acids, carotenes, and flavonoids. The term “biological active” in relation to compositions for cosmetic, cosmeceutical or nutriceutical applications also includes activity relating to the improvement of the outer part as well as the inner part of the body, in particular of the dermis and mucus membranes, as well as the general well-being of an individual.

In one embodiment of the invention, the active used in the invention will have low solubility in water, oils or organic solvents. In another embodiment the actives are poorly soluble in both hydrophilic (e.g. water and aqueous media) and lipophilic media (e.g. oils, organic solvents, liquid paraffin etc.). The porous particles may be dispersed in water, oils or organic solvents to increase the solubility of actives in these media (e.g. in oils for dermal application, e.g. baby oils).

In a preferred embodiment of the invention, actives useful in accordance with the invention include any low soluble active capable of being delivered by the topical route, e.g. the skin and/or mucosa. Such actives may be include, but are not limited to, pharmaceutical actives (drugs), cosmetic or cosmeceutical actives as described herein above. It is also within the scope of the invention that the active also includes nutraceutical actives capable of being delivered by the topical route.

In one embodiment of the invention, poorly soluble compounds or compounds with unsatisfying or low solubility useful in the present invention comprise pharmaceutical actives (drugs). Suitable pharmaceutical active include, but is not limited to the following:

Nonsteroidal anti-inflammatory drugs such as salicylates (e.g. diflunisal, salsalate), propionic acid derivatives (e.g. naproxen, oxaprozin), acetic acid derivatives (e.g. diclofenac, indomethacin, etodolac), enolic acid derivatives (e.g. piroxicam, lornoxicam), anthranilic acid derivatives (e.g. mefenamic acid, flufenamic acid), selective COX-2 inhibitors (e.g. firocoxib), sulfonanilides (e.g. nimesulide) and various other anti-inflammatory drugs (e.g. licofelone);

Reverse-transcriptase inhibitors such as e.g. nucleoside analog reverse-transcriptase inhibitors (e.g. zidovudine, stavudine, entecavir), nucleotide analog reverse-transcriptase inhibitors (e.g. tenofovir, adefovir), non-nucleoside reverse transcriptase inhibitor (e.g. nevirapine, efavirenz, rilpivirine);

Antibiotics such as ansamycins (e.g. rifaximin), carbacephems (e.g. loracarbef), carbapenems (e.g. doripenem, ertapenem, meropenem), cephalosporins (e.g. cefazolin, cefuroxime, ceftriaxone), lincosamides (e.g. clindamycin, lincomycin), macrolides (e.g. azithromycin, erythromycin, telithromycin), monobactams (e.g. aztreonam), nitrofurans (e.g. furazolidone, nitrofurantoin), oxazolidonones (e.g. linezolid), penicillins (e.g. amoxicillin, polypeptides (e.g. bacitracin), quinolones (e.g. levofloxacin), sulfonamides (e.g. sulfamethoxazole), tetracyclines (e.g. tetracycline.);

Peptides such as e.g. cyclic nonribosomal peptides (e.g. ciclosporin) and peptide hormones;

Corticosteroids such as glucocorticoids (e.g. prednisolone, hydrocortisone, dexamethasone, prednicarbate) and mineralocorticoids (e.g. aldosterone);

Aromatase inhibitor, i.e. non-selective (e.g. aminoglutethimide) and selective inhibitors (e.g. anastrozole); and

Antifungal drugs such as polyene antifungals (e.g. amphotericin B, nystatin), imidazole, triazole and thiazole antifungals (e.g. oxiconazole, abafungin), allylamines (e.g. naftifine, terbinafine), echinocandins (e.g. anidulafungin, caspofungin) and others (e.g. griseofulvin, tolnaftate).

In another embodiment of the invention, poorly soluble compounds or compounds with unsatisfying or low solubility which are useful in the present invention comprise non-pharmaceutical actives, such as for example, cosmetics, cosmeceuticals, nutraceuticals, such as for example:

Quinones, such as 1,4-benzoquinones (e.g. coenzyme Q10). Flavonoids such as e.g. anthoxanthins (e.g. quercetin, lutelin, apigenin, baicalein), flavanones (e.g. hespertin, hesperidin, naringenin,), flavanonols (e.g. dihydroquercetin, dihydrokaempferol), flavans (e.g. thearubigin); Carotinoids, i.e. carotenes (beta-carotene, alpha-carotene, beta cryptoxanthin, lycopene) and xanthyphylls (e.g. lutein, zeaxanthin, neoxanthin, violaxanthin);

Stilbenoids such as stilbenoid aglycones (e.g. resveratrol) and dihydro-stilbenoids (e.g. dihydro-resveratrol); and

Sun screens such as e.g. avobenzone. e, oxybenzone, octyl methoxycinnamate, octocrylene, octyl methoxycinnamate, apigenin, coenzyme Q10, quercetin, etc. . . .

In some cases sunscreens are desired to penetrate into the skin. Damage to the skin is caused by ultraviolet (UV) radiation, but also by infra red (IR) radiation. IR radiation can pass the sunscreen cream layer, penetrates deeply into the skin and can cause damage via generating free radicals (oxidative stress). To protect against IR radiation, sunscreens with antioxidative effect (e.g. apigenin) need to penetrate into the skin, which make the use of penetration enhancing porous material useful for skin protection.

Porous particles useful in the present invention may be organic or inorganic particles. In one embodiment of the invention, the porous particles are porous inorganic particles. Suitable porous materials include any porous particle which are chemically inert to a) any active to be used and b) body fluids of humans and animal. The porous particles may have a variety of different symmetrical, asymmetrical or irregular shapes, including chain, rod or lath shape. The particles may include mixtures of particles comprising different compositions, sizes, shapes or physical structures.

In a preferred embodiment of the invention, the porous particles are inorganic oxide particles. In one embodiment, the porous inorganic oxide particles comprise porous silica and silicates, e.g. magnesium-alumina silicate. Useful silica particles comprises, but are not limited to, precipitated silica, silica gel, fumed silica, colloidal silica, and combinations thereof, such for example, those silica sold by W. R. Grace & Co.—Conn., in Columbia, Md., under the tradename Syloid®, Aerosil®/Aeroperl®/Cab-o-Sil® (fumed silica base), Sylysia/Partec® SLC (silica gel), Perkasil® (precipitated silica).

Silica particles useful in the present invention may be comprised of both amorphous and crystalline structures and the pores can be polydisperse (i.e. non-ordered porous materials) in the pore diameter, or rather uniform in size (substantially uniform or “ordered porous material”) as in silica produced by the company FORMAC Pharmaceuticals N.V. Gaston Geenslaan 1, 3001 Leuven, Belgium), so called CMO technology by Formac. These silicas are as described e.g. in various patents/patent applications (e.g. EP2170289A2, EP2170289B1, U.S. Pat. No. 8,216,495, WO2009118356A2, WO2009118356A3, US20110018154A1, Preparation method for solid dispersions, inventors: Sandrien Janssens, Guy Van Den Mooter); CA2721485A1, CA2721485C, CN102066256A, EP2282973 A2, WO2009133100A2, WO2009133100A3, US20110081416 A1, Ordered mesoporous silica material, inventors: Jasper Jammaer, Alexander Aerts, Guy Van Den Mooter, Johan Martens; EP2646005A1, WO2012072580A1, US20130243833A1, Compressed formulations of ordered mesoporous silicas, inventors Monica Vialpando, Johan Martens, Guy Van Den Mooter, Filip Kiekens).

The silica particles may also comprise the so called “bimodal silica” by Merck Millipore (Frankfurter Straβc 250, Darmstadt, Germany), containing mesopores (2-50 nm) but also additional macropores with a size of e.g. about 2 μm, the silica having a large surface area (e.g. around 1000 m²/g) (Parteck® SLC silica). The silica can be made as granulate, e.g. with a particle size 5-25 μm (bimodal silica: a game-changing ingredient, H. Leonhard Ohrem and Roger Weibel, manufacturing chemist, page 28-29, Dec. 2012).

Silicas useful in the present invention can be made by basically two methods: Precipitation/gelation from solutions (wet process) and pyrolysis (dry process). The “wet process” comprises various synthesis routes including but not limited to precipitation (Ullmann Volume A 22 Silica, 642-647, VHC-Verlagsgesellschaft mbH, D-69451 Weinheim, 1993), colloidal formation (Ullmann Volume A 22 Silica, 614-629, VHC-Verlagsgesellschaft mbH, D-69451 Weinheim, 1993), gelation (Ullmann Volume A 22 Silica, 629-635, VHC-Verlagsgesellschaft mbH, D-69451 Weinheim, 1993) and electro-dialysis (U.S. Pat. No. 4,508,607). The “dry process” (Ullmann Volume A 22 Silica, 635-642, VHC-Verlagsgesellschaft mbH, D-69451 Weinheim, 1993) is in contrast to the “wet process” a high temperature process. With the exception of gelation all other silica making technologies create in the first reaction step building units of 10⁻⁹ meter to 10⁻⁶ meter size, which have to be aggregated and/or agglomerated in subsequent process steps. Such particle accumulation can be achieved via filtration and wet compaction, filter drying, reaction spray drying, spray drying, flash drying. The gelation process starts with the formation of a meter sized polymer, which has to be downsized by crushing and milling and subsequently dried. Drying can be achieved by but not limited to slow drying in stationary or rotary kilns or by fast drying in an expanding fluidized bed (flash drying) or in a jet mill energized with a hot gas, preferably steam or hot air. Such gel particles have an intrinsic pore structure, which can be tuned via time-, temperature- and pH-control.

Silica useful in the present invention may also contain metal ions in order to modify the silicas' physical, chemical and surface chemical characteristics. Typical ions include, but not limited to, alkali metals, earth alkali metals, transition metals, post transition metals, metalloids and combinations thereof The concentration of metal ions comprised in the silica can typically be 50 wt % or less (on an oxide basis) of the total silica composition. In one embodiment, the metal ion is present in a concentration up to about 80 wt % (on an oxide basis) of the total silica composition. In a preferred embodiment of the invention, the metal ion concentration ranges from about 1 to about 30% of the total silica concentration. The single building units from the “wet process” are known to be pore-free. Compacted silica made up by these building units show porosity, which is created by voids between individual building units. Porosity is prone to adsorption and may happen when a) the geometrical dimensions of adsorbent (silica) and adsorbate (pharmaceutically active material) are in line and b) there is an affinity between adsorbent and adsorbate. The latter is given when the surface of the silica has a terminal silanol group (Si—OH) density of approximately 5 per nm² (Ken K. Qian and Robin H. Bogner: Application of Mesoporous Silicon Dioxide and Silicate In Oral Amorphous Drug Delivery Systems. Journal of Pharmaceutical Sciences, Volume 101, Issue 2, pages 444-463, February 2012). These terminal silanol groups play a major role in silica-drug interaction during amorphization.

In one embodiment of this invention, the porous particulate material comprise an amorphous silicon dioxide. In a preferred embodiment, the silicon dioxide is one having specifications in accordance with the specifications of the United States Pharmacopoeia-National Formulatory (USP-NF) for Silicon Dioxide, the Japanese Pharmaceutical Excipients (JPE) for Hydrated Silicon Dioxide and the European Pharmacopoeiam (EP) for Colloidal Hydrated Silica, the definitions as being in force on 1 Sep. 2014.

It is also within the scope of this invention that porous particles to be loaded with the desired actives may comprise particles of an organic or inorganic nature, having the following features: a) the particles are inert to both any to be adsorbed and desorbed pharmaceutical active and any liquids of the human or animal body and b) the particles have an affinity to the active adsorbed therein or thereon. Suitable organic particles include natural (e.g. cellulose and its derivatives, polysaccharides, chitosan, hyaluronic acids, etc.) and synthetic polymers (e.g. from lactic acid, glycolic acid, polyhydroxybutyric acid, polymethylmethacrylates, polyurethanes, polycyanoacrylates, polyethylene etc.).

In one embodiment, porous particulate materials useful in the compositions of the invention have a specified pore diameter (PD)(nm) and specific surface area, SA, [m³/g]. These parameters are determined with the BET-Nitrogen absorption method (ISO 9277:2010). The semi empiric Wheeler formula PD=PV/SA*4000, (Elliott P. Barrett, Leslie G. Joyner, Paul P. Halenda, The Determination of Pore Volume and Area Distributions in Porous Substances: I. Computations from Nitrogen Isotherms. J. Am. Chem. Soc., Vol. 73, No. 1. 1 Jan. 1951, pp. 373-380), teaches that these parameters are not arbitrary.

In general, porous particulate materials used to prepare compositions of the present invention comprise a pore volume of 0.1 cm³/g or greater. In a preferred embodiment, the porous inorganic oxide material has a pore volume of about 0.5 cm³/g or greater, or about 0.6 cm³/g or greater, or about 0.7 cm³/g or greater. In some embodiments, the upper limit of the pore volume is about 3.0 cm³/g, or about 2.3 cm³/g.

Generally, the porous particles will typically have an average pore diameter of greater than or equal to 2 nm, or from about 2 to about 250 nm, or from about 2 to about 200 mn, or from about 2 to 100 nm. In a further embodiment, the particles have an average pore diameter from about 2 nm to about 50 nm or from about 5 to 40 or from 10 to 30 nm. In another embodiment the particles have an average pore diameter from about 50 nm to about 250 nm, or 60 to 200 nm, or about 80 to 150 nm.

The porous particulate material generally has a BET surface area, as measured by nitrogen adsorption, of about 10 m²/g or greater, or about 100 m²/g or greater, or of about 200 m²/g or greater, or of about 300 m²/g or greater. In some embodiments, the upper limit of the BET surface area is about 1000 m²/g, or about 800 m²/g, or of about 600 m²/g. In other embodiments, the BET surface area may range from about 10 to about 1000 m²/g, or about 100 to about 800 m²/g, or about 150 to about 600 m²/g, or about 200 to about 500 m²/g, or about 250 to about 400 m²/g.

The particle size of the porous particles will vary depending on the intended use of the loaded particles. The particle size is typically measured by laser diffraction using laser diffractometer (typically Mastersizer®, Malvern Instruments, United Kingdom), and calculated using the Fraunhofer theory, or alternatively the Mie theory. The sizes specified are the diameters 50%, i.e. average particle size.

Generally, the average particle size of the porous material is in the range of about 1,000 μm or less. In one embodiment, the average particle size ranges from about 0.1 μm to about 1,000 μm. In one embodiment of the invention, the porous particles have an average particle size of less than 125 μm or less than 63 μm, or less than 45 μm, or less than 24 μm, or less than 12 μm. In another embodiment, the porous particles have a particles size ranging from about 0.1 to about less than 125 μm for topical or dermal products. In a preferred embodiment, the porous particles have a particles size ranging from greater than 50 μm to less than about 125 μm for topical care products. In one embodiment, where occlusive or more abrasive topical formulations are desired, such as for example skin masks, the porous particles will have a particle size ranging from about 125 μm or greater to about 1,000 μm, preferably from about 150 μm to about 500 μm.

Loaded particles according to the invention can be obtained in principle, by any conventional method described in the literature for loading porous materials with an active provided however, that such loading method provide the active in an substantially amorphous or partially amorphous form of the active to be loaded. Such method includes, for example:

A. Wetness impregnation method: Active solution is added to the porous material under blending, and then the solvent is evaporated. This step can be repeated several times until the desired loading has been reached. The stepwise addition allows preferable filling of the pores, especially when using low concentrated solutions of active (Müller, R. H., Wei, Q., Keck, C. M., Stability of Industrially Feasible Amorphous Drug Formulations Generated in Porous Silica. Abstract W5313, AAPS Annual Meeting, San Antonio, 10-14 Nov. 2013).

B. Fluidized bed impregnation: Drug solution is sprayed into a fluidized bed dryer, which contains the porous materials in the fluidized bed. The solution droplets get in contact with the carrier and being adsorbed into the pores. The solvent is evaporated in the fluidized bed dryer, multiple loading and drying is possible (F. J., B. J. Glasser, and P. I. Gregorov, Formulation and Manufacture of Pharmaceuticals by Impregnation onto Porous Carriers, US20130236511A1, 2013, Rutgers, The State University of New Jersey).

C. Immersion method: The porous material is suspended in a drug solution, the pores fill, and then the porous material is separated from the solution (e.g. by sedimentation, centrifugation, filtration) and the solvent from the pores evaporated (e.g. compartment dryer, vacuum dryer etc.) (Zhai, Q. Z., Y. Y. Wu, and X. H. Wang, Synthesis, Characterization and Sustaining Controlled Release Effect of Mesoporous SBA-15/ramipril Composite Drug. J Inel Phenom Macro, 2013. 77(1-4): p. 113-120).

D. Super-critical CO₂ method: The mixture of porous material and drug are immersed in supercritical carbon dioxide and blended, the drug dissolves, removal of the super-critical state of carbon dioxide and evaporation leads to drug precipitation in the pores (Li-Hong, W., et al., A Novel Strategy to Design Sustained-Release Poorly Water-Soluble Drug Mesoporous Silica Microparticles Based on Supercritical Fluid Technique. Int J Pharm, 2013. 454(1): p. 135-42).

E. Melting Method: This method is solvent-free. Molten active is added to the porous material and blended, the melted drug adsorbs into the pores. Then the mixture is cooled (Aerts, C. A., et al., Potential of Amorphous Microporous Silica for Ibuprofen Controlled Release. Int J Pharm, 2010. 397(1-2): p. 84-91).

The technical advantage of using porous materials is that production can be accomplished in a one-step process, e.g. in a topogranulator using the wetness impregnation method or in a fluidized bed dryer using also the impregnation method.

Loading the particles with the active can be performed using solutions of the active in a suitable solvent (e.g. ethanol, methanol, isopropanol, dimethylsulfoxide (DMSO) etc.). i.e. using 2 compound systems. In one embodiment, additional excipients (i.e. solution additives) can be added (multiple compound systems), e.g. surfactants (e.g. Tween 80, examples 15 and 16), polymers, gelling agents or hydrophobic compounds. The surfactant may increase the wettability, thus accelerating release and dissolution. Polymers can modulate the release depending on the polymer used (e.g. hydrophilic polymers (Poloxamers polyethyleneglycol-propyleneglycol co-polymers) promoting release or viscous polymers delaying it (e.g. high molecular weight polyvinyl alcohol—PVA). Gelling agents make the fluids in the pores more viscous (e.g. xanthan gum), thus prolonging release. Addition of one or more excipients can be exploited to modulate the release.

Examples of solution additives include, but are not limited to, surfactants: anionic (e.g. sodium stearate; sodium dodecyllbenzene sulfonate), cationic (e.g. laurylamine hydrochloride, trimethyl dodecyl ammonium chloride) and nonionic surfactants/stabilizers: polyoxyethylene glycol alkylphenyl ethers (e.g. Triton® X-100), glycerol alkyl esters (e.g. monolaurin), sorbitan alkyl esters (Spans), cocamide monoethanolamine, dodecyldimethylamine oxide, block copolymers of polyethylene glycol and polypropylene glycol (poloxamers), polyethoxylated tallow amine, alkylphenol ethoxylates, alkyl polyglycoside (e.g. Plantacares), tocopheryl polyethylene glycol 1000 succinate (TPGS), polysorbates (Tweens). Also zwitterionic surfactants can be used (e.g. lecithin, lauramidopropyl betaine, dodecyl betaine, cocamidopropyl hydroxysultaine).

Polymers can be used such as e.g. copolymers of polyoxypropylene and polyoxyethylene (e.g. Poloxamers, Poloxamer 188, Poloxamer 407), polyethers (e.g. polyethylene glycol, polypropylene glycol, copolymers (e.g. poly(lactic-co-glycolic acid)), polyvinylesters (e.g. polyvinyl acetate, polyvinylpyrrolidone), polysaccharides (e.g. tragacanth, chitosan), cellulose derivatives (e.g. hydroxyethyl cellulose, hydroxypropyl cellulose, hydroxyethyl methyl cellulose, hydroxypropyl methyl cellulose), polyacrylic acids (e.g. Carbomer 940), polyvinyl alcohols.

In accordance with the invention, the active is preferably loaded and maintained substantially or partially in the amorphous state to provide the beneficial penetration enhancing effect. Formation of crystalline active does not provide this enhanced effect when loading porous particles since crystalline active has no increased saturation solubility. When loading porous structures with actives, firstly evaporation of solvent begins immediately after addition of the solution to the porous material. Secondly uptake of the solution into pores requires a quantity of time, preferably from about 0.1 minute to about 1 hour; plus time sufficient to permit the solvent to evaporate from the pores, e.g. from about 1 minute to about 10 hours depending on the particles, solution, evaporation temperature and pressure, and active used. Both effects, immediate start of evaporation and time for the solvent to penetrate into the pores, can lead to precipitation of amorphous active on the surface of the porous particles.

Further, addition of large quantities of solvent in relation to porous materials can leads to particles with crystalline drug, e.g. see US Patent Publication No. 20120076841A1 (Porous Particles Loaded with Cosmetically or Pharmaceutically Active Compounds; Simonnet, Jean-Thierry; Biatry, Bruno; Saint-Leger, Didier). In example 1, 20 g salicylic acid in 1 liter of acetone are added to only 200 g of porous silica (ratio of volume solvent to mass of porous carrier being about 5:1), in example 3, 2.5 g of triclosan dissolved in 50 ml of acetone are added to only 7.5 g of Orgasol® powder (ratio about 7:1), in example 4 1.5 g of vitamin E and 1 g of 5-n-octanoylsalicylic acid dissolved in 50 ml of acetone are added to 7.5 g of “God Balls 2 EC(R)” porous particles (again ratio about 7:1). Evaporation of solvent was performed at 40° C. Applying this described loading procedure lead to porous particles with crystalline drug (example 5). Thus care should be taken when loading the active containing solution to maintain the active in the amorphous state.

To maintain the active in a substantially or partially amorphous state when loading in solution, it is preferably to add the solution containing the desired active in small portions to ensure fast uptake by the pores and to prevent larger quantities of the active solution outside of the particles for a time where the solvent can evaporate and forms crystals. In a one embodiment of the invention, the active containing solution is loaded on the porous particles at a ratio of active solution to porous material of 1:1. In another embodiment of the invention, the active solution is loaded on the porous particles in a ratio of less than 1, or less than 0.9, or less than 0.8, or less than 0.7, or less than 0.6, or less than 0.5 to 1 (e.g. examples 1 to 3).

In one embodiment of the invention, a biological active can be loaded in a substantially or partially amorphous state by generating thin amorphous layers of active on the surface of the organic or inorganic particles. In this embodiment, the particles may be porous or non-porous. See, for example, the use of the non-porous Aerosil® 200 silica (table 2). The thickness of the layers on the particles will vary depending on such factors as the type of particles and the active used. In general the thickness of the active layer will be a thickness sufficient to maintain the active in a substantially amorphous or partially amorphous state. As will be understood by one skilled in the arts, maintaining the amorphous state depends on the compound-specific thermodynamic re-crystallization tendency and can be readily determined by analysis using x-ray diffraction or differential scanning calorimetry (DSC). In general, the thickness of the active will be a thickness less than a thickness exhibiting crystallization peaks on the x-ray diffraction (e.g. example 4, 33.3% loading with azithromycin, example 4a, loading with salicylic acid) or DSC.

The present invention also permit modulation of the release of the active in cases where a specified rate of penetration is not pharmacologically desired. In one embodiment, this can be achieved by the process of adding excipients which reduces the wettability of the loaded active. Examples of such excipients included, but are not limited to lipids (glyceride, oil or wax) or natural (e.g. petrolatum) or synthetic hydrocarbons. In another embodiment, modulation of release of the active may be accomplished by modifying chemically the surface of the pores inside the porous materials, such as, for example, by binding functional groups which specifically interact with the loaded active slowing down its release (e.g. introduction of functional group such as achievable by silanization).

In another embodiment of the invention, the active-loaded porous materials can be combined with nanoparticles, e.g. nanocrystals. The nanocrystals are generally too large (typically >100 nm or >200 nm) to be absorbed into the fine pores, being typically in the range less than 100 nm, or even 50 nm or smaller. However, the nanocrystals can be adsorbed onto the surface of the porous materials. This provides a dissolving depot on the particle surface. The nanocrystals can be adsorbed to porous particles being loaded with active. Alternatively, the nanocrystals can be adsorbed to unloaded porous particles, which are later admixed to a loaded porous particle to “fine tune” a release profile. Alternatively to nanocrystals, lipid nanoparticles with solid particle matrix, e.g. solid lipid nanoparticles (SLN) or nanostructured lipid carriers (NLC) can be adsorbed, providing even more flexibility to control release, because SLN and NLC are matrix particles. The matrix allows one to adjust the release velocity, whereas in contrast nanocrystals without matrix material undergo straight dissolution. Instead of lipidic SLN and NLC, also liposomes can be used. Different nanoparticles can also be used in mixture, of two or more types.

When using nanoparticles, the loading may be performed by adding stepwise the nanosuspension (nanocrystals dispersed in liquid) or e.g. the SLN or NLC dispersion (typically aqueous but not necessarily) to the powder of the porous material under blending (lab scale: ointment bowl and pistil; large scale: granulators), and then the dispersion medium is evaporated. The particles remain adhered to the surface of the porous material.

Compositions in accordance with the invention may be incorporated into dermal and/or topical formulations using conventional methodology. Incorporation of the loaded particles into dermal or topical formulations may be accomplished using conventional methodology. The dermal or topical compositions may be in the form of creams, e.g. oil-in-water creams, pastes, serums, gels, lotions, oils, milks, sticks, ointments, solutions, suspensions, dispersions, or emulsions. Depending on the end use, the compositions are incorporated in an amount sufficient to provide biological activity, i.e. cosmetic, cosmeceutical, pharmaceutical or the like, when applied to the skin and/or mucous membrane in humans and animals.

Typically, the porous materials are dispersed in water by high sheer agitation for preparation of gels or creams. For example for preparation of a gel, all excipients/actives and the porous material are dispersed in the water phase and then the gelling agent is added. For preparation of an oil-in-water cream, other excipients such as surfactants are added to the water containing the porous material, and then the oil phase is added and dispersed by stirring. Dermal and topical formulations may also be prepared by admixing the powder of porous particulate materials after production of a gel or cream in a final production step, preferentially at low temperature of 30-40° C. or at room temperature. Here, an advantage is that incorporation of the loaded porous materials in the final formulations can be accomplished using existing production lines.

Excipients for preparing the gels include but are not limited to Poloxamers (e.g. poloxamer 188, poloxamer 407), polysaccharides (e.g. tragacanth, chitosan), cellulose derivatives (e.g. hydroxyethyl cellulose, hydroxypropyl cellulose, hydroxyethyl methyl cellulose, hydroxypropyl methyl cellulose), starch and starch derivates, alginates, polyacrylic acids (e.g. carbomer 940), silicas (e.g. Aerosil® 200 silica), gelatin and bentonite.

Topical and dermal formulations may also be prepared by dispersing the loaded porous materials of the invention in a phase with higher viscosity, e.g. a semi-liquid phase (e.g. viscous oils, vaseline, petrolatum jelly). The viscosity of this phase can be relatively high (semisolid) to very high, i.e. the phase is a solid matrix, e.g. the polymer matrix of a dermal patch (e.g. made from polymers based on acrylic esters such as 2-EHA (2-ethylhexyl acrylate) and ethyl acrylate (DURO-TAK® adhesive from the company Henkel, Germany) or polyurethane patches from Otto Bock, Germany (US 2011/0229677A1, Polyurethane Plaster for the Transdermal Application of Active Substances, and Method for the Production Thereof; Gansen, Peter; Dittgen, Michael; Steinfatt-Hoffmann, Ingeborg; Chulte, Christian; Henze, Juergen; Keck, C. M., Gansen, P., Bohrer, B., Müller, R. H., Dittgen, M., High tolerance transdermal patches loaded with caffeine, #899, Int. Symp. Control. Rel. Bioact. Mater. 40, Honolulu/Hawaii, 21-24 Jul. 2013). The porous material increases skin penetration, in addition leads the occlusive effect of the patch to an additional penetration enhancement.

One type of dermal patch useful in the present invention is a pre-formed dermal patch. Pre-formed dermal patches comprise films to be applied to the skin. In one embodiment, the patches, so called in situ forming patches, are formed from film forming emulsions which form the film after application to the skin (e.g. Dominique Jasmin Lunter, Rolf Daniels, New Film Forming Emulsions Containing Eudragit® NE and/or RS 30 D for Sustained Dermal Delivery of Nonivamide. European Journal of Pharmaceutics and Biopharmaceutics, 82, 291-298, 2012 and; Dominique Jasmin Lunter, Rolf Daniels, In vitro Skin Permeation and Penetration Nonivamide from Novel Film Forming Emulsions. Skin Pharmacology and Physiology, 26, 139-146, 2012). These emulsions consist typically of an oil phase, and a water phase (typically one of them containing the active), stabilizer and/or viscosity enhancer plus water-insoluble polymeric particles. The particles may comprise various polymethacrylate-polymers (e.g. Eudragit®), or other polymers with a size allowing, after evaporation of the water, a film formation on the skin (i.e. sufficient film forming capillary forces). Water soluble actives may be incorporated in the water phase, and lipophilic, oil soluble actives in the oil phase. Porous particles may be used to load actives which may be simultaneously poorly soluble in water and in oils. Active-loaded porous particles, preferentially inorganic porous particles such as silica, may be added to these in situ forming patches with a film forming mixture. Instead of loading the oil with lipophilic actives, these lipophilic actives may also be loaded into the porous particles, which increases their dermal bioavailability compared to simple incorporation into the oil phase.

In another embodiment, in situ forming patches with film forming dermal formulations may be prepared using the system described above, but without oil phase, i.e. aqueous suspensions of polymeric particles. Active-loaded porous particles may also be admixed with these suspensions. Many porous particles may affect the properties of dermal formulations such as viscosity and spreadability. It is also within the scope of the invention to add in addition to active-loaded porous particles also a fraction of unloaded particles. The unloaded particles may affect also the structure of the formed film, e.g. porosity, and thus the release of the active. Therefore addition of unloaded porous particles may also be preformed to modulate the release.

In yet another embodiment, the loaded porous materials of the invention may be used in a wafer. Similar to dermal pre-formed patches, topical and dermal or mucosal formulations may be prepared by dispersing the loaded porous materials into the solid phase of a wafer. Examples of wafers useful in the present invention are as described in Papola Vibhooti, Kothiyal Preeti, Wafers Technology—A Newer Approach to Smart Drug Delivery System, Indian Journal of Research in Pharmacy and Biotechnology. Volume 1(3) May-June 2013 Page 428; and in Boateng J S, Matthews K H, Auffret A D, Humphrey M J, Stevens H N, Eccleston G M, In Vitro Drug Release Studies of Polymeric Freeze-Dried Wafers and Solvent-Cast Films Using Paracetamol as a Model Soluble Drug. Int J Pharm. 2009 Aug. 13; 378(1-2):66-72. doi: 10.1016/j.ijpharm.2009.05.038. Epub 2009 May 27). The loaded porous material may be incorporated into different types of wafers, e.g. flash dissolved wafers, melt away wafers, sustained release wafers and flash dispersed wafers. The wafers may be produced conventional methods, e.g. via lyophilisation or solvent-casting. The preferential route of administration of the water containing the loaded porous material is oromucosal, i.e. through the mouth cavity, but application to other mucosal surfaces is also possible. The wafers may also be applied to the skin, e.g. facial treatment in cosmetics with cosmetic actives. Other wafers useful in the present invention include the oral medical wafers of the Company LTS Lohmann Therapie-Systeme AG (Andernach, Germany). The wafers may be flash release wafers, mucoadhesive melt-away wafers and mucoadhesive sustained release wafers. The size of the wafers typically varies between 2 cm² and 8 cm² area with a thickness between 20 μm and 500 μm. From this the porous materials can easily be incorporated in these films. Places of application in the mouth include e.g., the tongue, gingival, teeth, buccal region, or upper palate. The drug action may be systemic or local.

When incorporated into dermal formulations or being applied to the skin (comparable when using sun lotion on a beach with sand grains on the skin contaminating the sun lotion), particles in the micrometer size range can create a sandy feeling. A full range of different porous materials was incorporated at different concentrations in a gel and the skin feeling tested (example 10). For most of the materials no sandy feeling was observed, that means they are suitable for dermal formulations. In particular, the Syloid® silica formulations had a pleasant feeling on the skin.

In one embodiment, silica particles useful in the present invention should have a particle size of less than 125 μm for having a pleasant skin feeling in skin products (preferably range 0.1 μm to less than125 μm), most preferable, less than 63 μm, even more preferably, less than 45 μm, most preferable less than 24 μm and ideally less than 12 μm (Table 2) depending on the intended use. For example, for skin care products, since skin feeling of silicas embedded in creams, lotions etc., for topical usage is of importance, the particle size of the silica according to this invention should be less than 125 μm for skin care products. Both irregularly shaped and more spherical porous materials can be used. The latter allows the use of larger size particles without causing a sandy skin feeling so in general a particle size can be up to 30% larger that the former.

The loaded porous materials may also be used in facial (topical) masks, to combine the effect of a dermal active with the peeling effect of such masks, or the occlusion or other effects of masks. In masks (e.g. peeling masks to achieve a pronounced peeling effect) the porous material should have a mean particle size of greater than 125 μm, containing particles up to about 500 μm, and a maximum up to about 1,000 μm.

The drug delivery properties of the porous particles of the invention for topical administration may also be exploited for mucosal delivery, such as for example, oromucosal delivery in the oral cavity and pharynx. Apart from being used as suspension, a spray, or incorporated in mucosal films and patches, the loaded porous particles of the invention can be incorporated into oromucosal formulations, such as for example, but not limited to, lozengers, oral disintegrating tablets (ODT), oral gels and creams, and also into chewing gum or other oromucosal formulations.

In one embodiment, oromuscosal delivery may be accomplished using a chewing gum formulation. Typically, chewing gum formulations comprise a gum base matrix with excipients to provide the required masticatory and other sensory characteristics for the consumer. The gum base matrix may comprise at least 5% up to about 97% of the gum formulation. In a preferred embodiment, the gum base matrix comprises above 25% (weight/weight), for example 30%, 35%, 40% or up to 50% of the gum formulation. A typical chewing gum base also comprises components, including but not limited to, elastomers, softeners, emulsifiers, resins, polyterpenes, waxes (e.g. paraffin, microcrystalline wax), fats (e.g. hydrogenated oils) and mixtures thereof. In a preferred embodiment, the chewing gum formulation comprises a mixture of at least two of these components. Elastomers suitable in gum formulation include, for example, natural latexes (e.g. couma macrocarpa (i.e. leche caspi or sorva), loquat (i.e. nispero), tunu, jelutong, or chicle), or synthetic rubbers (e.g. styrene-butadiene rubber, butyl rubber, or polyisobutylene). Additional excipients useful in gum formulations include, for example, but are not limited to, flavours (e.g. menthol, peppermint) and stabilizers (e.g. antioxidants). The porous particles may be incorporated directly into the gum base matrix, or may be admixed with the excipients and added to the gum base matrix to yield the chewing gum formulation. During chewing, a supersaturated solution is formed in the mouth cavity and/or pharynx which solution comprises the actives to be delivered into the body through the mucosa of the oral cavity.

In another embodiment, topical application may be accomplished through the nasal cavity. Application to the upper nasal cavity can be used to achieve brain delivery of actives. The porous particles may be administered, for example, but not limited to, in the form of a nasal cream (oil-in-water cream), ointment, gel, nasal drops, a nasal spray (i.e. a suspension of the porous particles dispersed in a liquid), powder spray (porous particles in gas phase), or dispersed in a nasal tampon. Preferred particle size of the porous particles for nasal delivery is less than 50 μm, more preferably less than 10 μm and most preferred less than 5 μm. In the most preferred embodiment, the particle size of the porous particles for nasal delivery is less than 2 μm. Reduction in size increases mucoadhesion. In addition, a mucoadhesive coating can be applied onto the porous particles (e.g. chitosan polymer, polyvinyl alcohol (PVA), gum arabic, or block-copolymers of polyoxyethylene-polyoxypropylene type (e.g. products Poloxamer, Pluronic).

In yet another embodiment, topical application of the loaded porous particles of the invention may be accomplished by application into the eye for ocular delivery. In this embodiment, the porous particles may be applied to the eye as eye drops in the form of a liquid suspension. To minimize eye irritation, the particles size of the porous particles should be less than 10 μm, preferably less than 5μm, more preferably less than 1 μm and even more preferably, less than 1 μm. Alternatively, the loaded porous particles the invention may be applied to the eye incorporated into a gel, a self-gelling gel, a cream or an ointment. The loaded porous particles of the invention may also be delivered into the eye by incorporation into inserts, such as for example, by incorporation into eye contact lenses or implants for injection into the eye. From the injection site, the actives released can diffuse into the surface of the eye. For injectable formulations, the porous particles should ideally be degradable in the body.

It is also within the scope of this invention, that dermal delivery can be further enhanced by accumulation of the means for porous particles in the hair follicles. Particles resting in the hair follicles act as a means for releasing the active over a longer period of time. In addition, the deep follicles can better penetrate the active into the surrounding cells than the skin surface. To access the hair follicles the size of the porous particles should be less than 20 μm, preferred less than 5 μm, more preferred less than 2 μm and optimal less than 1 μm, to reach the deeper follicles. By doing this, hair follicle targeting formulations are available. The formulation may be massaged into the skin, to enhance the localization of the porous particles in the hair follicles. To make massaging possible, the formulation should have a sufficiently low viscosity (preferably less than viscous petrolatum, United States Pharmacopeia).

The compositions in accordance with the invention provided strong drug delivery properties for topical and dermal formulations. The compositions provide superior penetration into the skin and mucosa as compared to microcrystals of the active, and surprisingly also compared to nanocrystals in the dermal formulation (Example 12). Theoretically higher penetration would have been expected from the nanocrystals due to their much larger surface area of the active (surface of nanocrystals) being in contact with the water phase (=fast dissolution) compared to the active in the pores of the porous material (much smaller cross sectional area of pores in contact with water phase). Further, one would expect that where the active is loaded on the surface of porous particles in accordance with the invention come in direct contact with the water, and subject to dissolution and re-crystallization phenomena to convert to the crystalline state. However, it was surprisingly observed that the surface-adsorbed active on the porous particulate materials remains amorphous.

Advantageously, the loaded porous materials of the invention offer other technical advantages compared to nanocrystals. The production is cheaper (current price of 50 g dermal nanocrystals by PharmaSol, GmbH Berlin about 1,000). Due to the high degree of dispersitivity (small size with high surface energy), the nanocrystals a priori are a thermodynamically instable system, with tendency to aggregate. Aggregated nanocrystals loose their special properties, e.g. high dissolution velocity. The micrometer-sized porous material tends less to aggregation, in addition aggregation has little or no affects on the status of the loaded active.

When the loaded particles in accordance with the invention are incorporated in the aqueous phase of dermal formulations (e.g. gels), surprisingly the amorphous state in the liquid dispersion medium remained stable (Example 11). Also formulations may be produced with a part of the drug being located on the surface of the porous particles by using higher concentrated impregnation solution. Even this amorphous surface layer in contact with the water does not crystallize. Based on this, the active can be loaded inside the pores, or partially inside the pores and/or outside on the surface on the porous particles.

The particle sizes given herein are typically measured by laser diffraction, as described above. Alternatively measurements can be performed using scanning electron microscopy (SEM), the diameter calculated D is the sum of largest dimension d1 and smallest dimension d2 of the particle divided by 2 equaling [ D=(d1+d2)/2].

To further illustrate the present invention and the advantages thereof, the following specific examples are given. The examples are given as specific illustrations of the claimed invention. It should be understood, however, that the invention is not intended to be limited to the specific details set forth in the examples. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

All parts and percentages in the examples as well as the remainder of the specification that refers to solid compositions or concentrations are by weight unless otherwise specified. However, all parts and percentages in the examples as well as the remainder of the specification referring to gas compositions are molar or by volume unless otherwise specified.

Further, any range of numbers recited in the specification or claims, such as that representing a particular set of properties, units of measure, conditions, physical states or percentages, is intended to literally incorporate expressly herein by reference or otherwise, any number falling within such range, including any subset of numbers within any range so recited.

### EXAMPLES

To investigate penetration of active from the porous material into the skin, an in vitro test using pig ear skin was performed. The drug azithromycin was selected. The relative penetration was assessed by tape stripping the stratum corneum. Azithromycin is an antibiotic in clinical phase 3 for dermal application to prevent borreliosis infection after tick bites. After the tick bite the parasites stay for some time at the place of bite. Dermal antibiotic application can kill the parasites in the skin and thus prevent infection. Pre-requisite is that there is a sufficient skin penetration of the antibiotic. The clinical test formulation as described in (Knauer, J. et al., J. Antimicrob. Chemother. 66 (12), 2814-2822, 2011) and nanocrystals were taken as reference. Penetration from the porous Syloid® silica was found to be superior to both reference formulations when considering the penetration into the deeper cell layers (higher number of strips) (example 12).

Nanocrystals dispersed in a dermal formulation (nano-suspension) are thermodynamically less stable compared to μm-sized suspension as the porous materials. As finely dispersed material they have a larger surface area and thus higher interfacial energy E (increase in surface energy E, E=A×γ, A—surface area, γ-interfacial tension). Ionic compounds typically present in dermal formulations decrease the zeta potential (=repulsive force between particles), thus further destabilizing the system. Nanosuspensions are more susceptible to a zeta potential decrease—compared to μm-sized suspensions—due to their higher diffusion velocity (diffusion constant D is proportional to particle size, Einstein equation). The porous material showed no aggregation in the gels (example 12).

### EXAMPLES

### Example 1

### Loading of Syloid® SP53D-11920 Silica With Azithromycin—Loading 32% (w/w)

First the drug azithromycin dihydrate raw powder was dissolved in ethanol (96%) in a ratio of 1:4 by weight to get azithromycin ethanol solution. Then 32.0% loading Syloid® SP53D-11920 silica was achieved by 3 steps.

In the first step, 2.5 g Syloid® SP53D-11920 silica was loaded with 0.5 g drug by addition of 2.5 g solution under stirring using an ointment bowl and pestle. To ensure that the drug solution was absorbed by the silica immediately and homogenously, the azithromycin solution was sprayed manually by a spraying nozzle screwed onto a glass bottle. Subsequently the ethanol was evaporated at 40° C. in a compartment dryer. The complete evaporation was controlled via determining the weight loss.

In the second step, 2.25 g of the obtained silica was loaded with 0.4 g drug by spraying of 2 g solution using the same method. In the third step, 2.025 g of this silica was loaded with 0.186 g drug by spraying of 0.93 g solution.

### Example 2

### Loading of Aeroperl® 300 Silica With Azithromycin—Loading 27.4%

The loading method was identical to example 1, but applying only 2 steps. Azithromycin was dissolved in ethanol (96%) in a ratio of 1:4 by weight to get azithromycin ethanol solution. Then 27.4% loading of Aeroperl® 300 silica was achieved by 2 steps. In the first step, 2.5 g Aeroperl® 300 silica was loaded with 0.5 g drug by spraying of 2.5 g solution onto Aeroperl® 300 silica under stirring using ointment bowl and pestle. In the second step, 2.25 g of the obtained silica was loaded with 0.4 g drug by addition of 2 g solution using analogous method.

### Example 3

### Loading of Neusilin® US2 Silica With Azithromycin

The loading method was identical to example 2. Azithromycin was dissolved in ethanol (96%) in a ratio of 1:4 by weight to get azithromycin ethanol solution. Then 27.4% loading Neusilin® US2 silica was achieved by 2 steps. In the first step, 2.5 g Neusilin® US2 silica was loaded with 0.5 g drug by spraying of 2.5 g solution under stirring using mortar and pestle. In the second step, 2.25 g of this silica was loaded with 0.4 g drug by spraying of 2 g solution.

### Example 4

### Determination of Maximum Amorphous Loading of Azithromycin Onto Syloid® SP53D-11920 Silica

Syloid® silica was loaded with increasing concentrations of azithromycin. The maximum loading was monitored by x-ray diffraction (XRD). Overloading was observed by detecting peaks of crystallinity in the x-ray spectrum, meaning that the drug is not more completely in the amorphous state. The samples were analyzed by placing a thin layer of the loaded silica powder in a Philips X-ray Generator PW 1830. The diffraction angle range was between 0.6°-40° with a step size of 0.04° per 2 seconds. The diffraction pattern was measured at a voltage of 40 kV and a current of 25 mA.

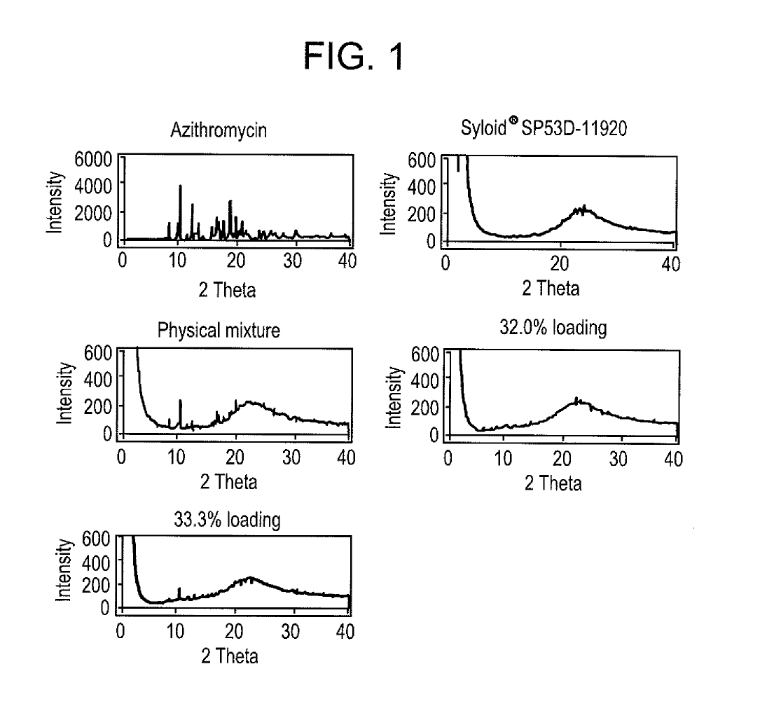

FIG. 1 shows the XRD patterns for crystalline azithromycin raw drug powder and that of pure amorphous Syloid® SP53D-11920 silica. Furthermore the spectrum of the physical mixture (=mixing Syloid® SP53D-11920 silica powder and 10% azithromycin raw drug powder with a mortar and pestle) showed crystalline peaks of azithromycin. In contrast the azithromycin loaded Syloid® SP53D-11920 silica (32.0% azithromycin) showed no crystalline peaks whereby loading of 33.3% azithromycin showed first small crystalline peaks, indicating the possible maximum loading of 32.0%. Besides the theoretical loading of 32.0%, the azithromycin content was measured by HPLC yielding a loading of 30.5% azithromycin.

### Example 5

### Loading of Porous Material With High Ratio Drug Solution to Porous Material

Formulations were directly prepared in the 40 mg pans for the differential scanning calorimetry (DSC, Mettler-Toledo, Germany), to ensure having a representative sample and correct calculation of melting enthalpies to quantify the degree of crystallinty. A total of 18.8430 mg (approx. 24 μl) acetone with 0.4770 mg dissolved salicylic acid was added to 4.7648 mg of porous SP53D-11920 silica (ratio volume solution to porous particles about 5:1) and mixed. After evaporation of the solvent, this corresponded to a drug-porous particle ratio of 1:10 (9.1% plus 90.9%). Evaporation was performed by at 75° C. (above boiling point of 56° C.) in a compartment dryer for 6 hours, which simulates the fast evaporation velocity at 40° C. in a rotary evaporator. As reference a physical mixture was prepared (also directly in the pan), mixing 2.3600 mg of salicylic acid with 23.3665 mg porous SP53D-11920 (25,934 mg in total). In the DSC apparatus, the samples were heated with a heating rate of 10K/min, from 25° C. to 180° C. and 200° C., respectively. The physical mixture was heated in a medium pressure pan (no evaporation of water) and the loaded porous particle in a normal punched DSC pan (with evaporation of potential residual solvent and respectively water).

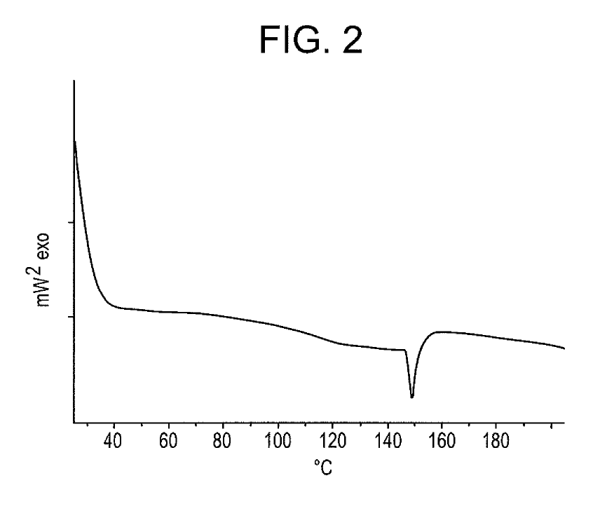

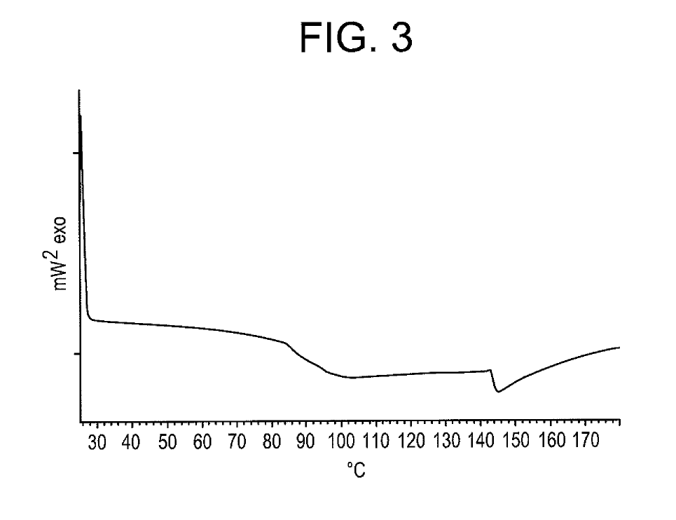

The physical mixture revealed a melting peak at 144.95° C. (FIG. 2), being below the 161.17° C. found for the pure drug. The solvent loaded porous particles yielded a drop in the base line between about 80° C. and 100° C. due to evaporation of water, and a melting peak at 148.83° C. (FIG. 3), proving the crystallinity. The melting enthalpies of the physical mixture and drug in solvent-loaded porous particles were 11.77 J/g and 19.37 J/g which proves the crystallinty of the loaded porous sample.

### Example 6

### Saturation Solubility of Azithromycin-Syloid SP53D-11920 Silica

The 32.0% azithromycin loaded Syloid® SP53D-11920 silica was dispersed in Milli-Q® water to get a final concentration of azithromycin of 4.8% in vials. 35.0% azithromycin physical mixture was as well dispersed in Milli-Q® water to get a final concentration of azithromycin of 5.6% in vials. Furthermore, 5.6% coarse drug powder was suspended in Milli-Q® water for comparison. The samples were stored at 25° C. shaking with 100 rpm in an Innova® 4230 shaker for 40 minutes. To separate the dissolved drug, the samples were first centrifuged (17,968×g; 10 minutes) and subsequently the supernatant was filtered (50 nm pore size, Whatman® 110603 filter). The drug concentration in such obtained sample was determined by HPLC.

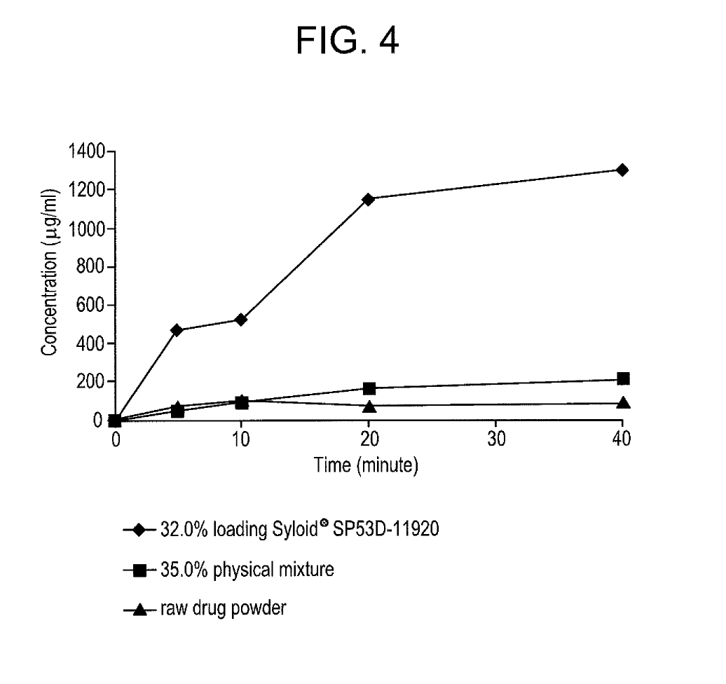

As shown in FIG. 4, the 32.0% loading Syloid® SP53D-11920 silica had an about 6 times higher saturation solubility in water (1300 μg/mL) compared to the physical mixture (213 μg/mL) and 14 times higher than that of raw drug powder (93 μg/mL) at 40 minutes.

### Example 7

### Preparation of Azithromycin Nanocrystals

10% azithromycin was dispersed in 1% tocopheryl polyethylene glycol 1000 succinate (TPGS) solution by Ultra-Turrax (Jahnke and Kunkel, T25) for 1 minute at 8,000 rpm. The resulting coarse suspension was wet milled using 0.1 mm diameter yttrium-stabilized zirconia beads using a bead mill, model PML 2 (Bühler, Switzerland), small milling chamber, at a rotation speed of 2000 rpm for 10 minutes. The process was performed at 5° C. by controlled circulation of cooled water through the outer temperature control jacket. The obtained particle size was 189 nm (zave), determined by photon correlation spectroscopy (PCS) using a Zetasizer® Nano ZS (Malvern Instruments, UK).

### Example 8

### Comparison of Saturation Solubility of Azithromycin Nanocrystals and Azithromycin Syloid® SP53D-11920 Silica

The azithromycin nanosuspension was dispersed in Milli-Q® water to get a final concentration of azithromycin of 2% in the vials. The samples were stored at 25° C. shaking with 100 rpm in an Innova® 4230 shaker for 60 minutes. Centrifugal ultrafiltration (molecular weight cut off 3000 Dalton) was chosen to separate undissolved drug nanocrystals. Subsequently HPLC measurements were performed to determine the concentration of dissolved azithromycin.

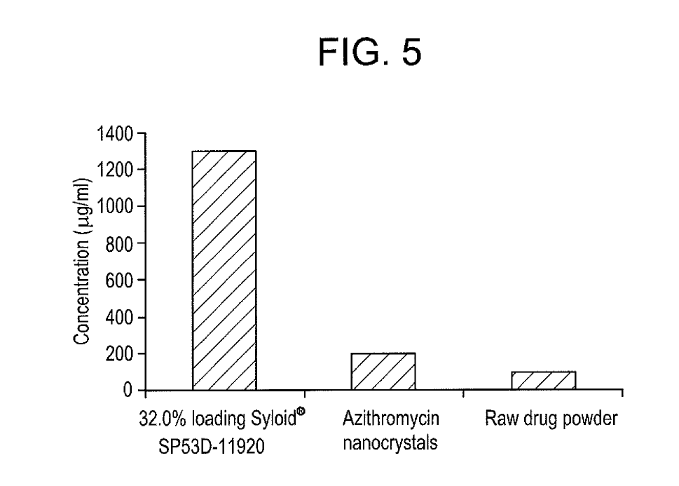

Samples were taken after 40 minutes (32.0% loading Syloid® SP53D-11920 silica) and after 60 minutes (azithromycin nanocrystals; raw drug powder). The nanocrystals (about 200 μg/ml) had about a 2 times higher saturation solubility Cs compared to the raw drug powder (95 μg/mL), the 32.0% loaded Syloid® SP53D-11920 silica had an about 6.5 times higher saturation solubility (1300 μg/mL) compared to the nanocrystals (FIG. 5).

### Example 9

### Saturation Solubility of Azithromycin-Loaded Neusilin® US2 Silica

Neusilin® US silica was loaded with azithromycin as described in example 2, applying 2 steps of loading (i.e. addition of 10% drug in ethanol, evaporation), yielding a loading of 26.4% (determined by HPLC). Saturation solubility was determined in water after 4 hours shaking, as described in example 8. The amorphous azithromycin in Neusilin® US2 silica had an about 25 times higher saturation solubility compared to raw drug powder.

### Example 10

### Preparation of Gels With Silica for Skin Feeling Testing

The basic recipe for preparation of the silica-containing gels was:

| | | |
|---|---|---|
| hydroxypropylcellulose (HPC), 70 kD | 0.0/5.0 | g |
| silica | 1.0/2.0/5.0 | g |
| Milli-Q ® water up to | 100.0 | g |

For the preparation of the gel base, Milli-Q® water was heated to 75° C. in an ointment bowl. Subsequently the HPC powder was added to the water and dispersed using a pestle until a homogenous suspension resulted. The mixture turned into a transparent gel base after storage overnight at 4° C. in the fridge. Overnight evaporated Milli-Q° water was supplemented at room temperature. To this gel base different kinds of silica (Table 1) were admixed by stirring manually with a pestle until the silica was uniformly dispersed into the gel base.

A series of formulations were produced containing 1%, 2% and 5% silica (w/w) in water (=aqueous suspension) and 1%, 2% and 5% silica (w/w) with 5% HPC (=gel). The silica suspensions and gels were transferred into glass vials, sealed and stored at 4° C. until they were examined regarding skin feeling the next day.

A series of formulations were produced containing 1%, 2% and 5% silica (wlw) in water (=aqueous suspension) and 1%, 2% and 5% silica (w/w) with 5% HIPC (=gel). The silica suspensions and gels were transferred into glass vials, sealed and stored at 4° C. until they were examined regarding skin feeling the next day.

TABLE 1
Type of silica, maximum concentration (Cmax) without
sandy feeling on skin in gel (middle) and as suspension
in water (right) (SF = sandy feeling).

| | Cₘₐₓ with 5% | Cₘₐₓ without |
|---|---|---|
| type of silica | HPC*(=gel) | HPC*(=aqueous suspension) |
| Aerosil ® 200 | 5% | 5% |
| Aeroperl ® 300 | 5% | 5% |
| Neusilin ® US2 | 1% | 1% |
| Neusilin ® SG2 | SF | SF |
| Syloid ® SP53D-11804 | 5% | 5% |
| Syloid ® SP53D-11920 | 5% | 5% |
| Syloid ® SP53D-11921 | 5% | 5% |
| Syloid ® SP53D-11922 | 5% | 5% |

*hydroxypropylcellulose, 70 KDa

TABLE 2
Properties of various Silica Materials

| | | | | | specific | |
|---|---|---|---|---|---|---|
| | | | | particle size | surface | pore |
| | | type of | average pore | distribution | area | volume |
| | producing company | porous | size (nm) | (μm) | (m²/g) | (cm²/g) |
| Aerosil ® 200 | Evonik Industries AG, | nonporous | / | | 200 ± 25 | / |
| | Hanau-Wolfgang, | | | | | |
| | Germany | | | | | |
| Neusilin ® SG2 | Fuji Chemical | porous | 1-2 | 125-500 | 110 | N/A |
| | Industry, Toyama, | | | | | |
| | Japan | | | | | |
| Aeroperl ® 300 | Evonik Industries AG, | porous | 2-50 | 30-40 | 300 | 1.6 |
| Pharma | Hanau-Wolfgang, | | | | | |
| | Germany | | | | | |
| Neusilin ® US2 | Fuji Chemical | porous | 5-6 | 60-120 | 300 | 1.2 |
| | Industry, Toyama, | | | | | |
| | Japan | | | | | |
| Syloid ® | W. R. Grace & | porous | 6 | 20-45 | 550 | 0.9 |
| SP53D-11804 | Co., Columbia, USA | | | | | |
| Syloid ® | W. R. Grace & | porous | 6 | 12 | 550 | 0.9 |
| SP53D-11920 | Co., Columbia, USA | | | | | |
| Syloid ® | W. R. Grace & | porous | 25 | 16-24 | 310 | 1.85 |
| SP53D-11921 | Co., Columbia, USA | | | | | |
| Syloid ® | W. R. Grace & | porous | 25 | 40-63 | 310 | 1.85 |
| SP53D-11922 | Co., Columbia, USA | | | | | |

To assess the skin feeling, an adequate amount of silica gel (ca. 20-40 mg/cm²) was applied on the underside of the wrist of volunteers (n=3) to check the skin feeling during application and rubbing in for about 1 minute. The results are shown in Table 1, an overview of properties of various silica materials is given in Table 2.

From the size data it can be concluded that materials with a size above 125 μm as Neusilin SG2 silica cause a sandy feeling on the skin, all materials with lower sizes created no negative skin feeling during application.

### Example 11

### Stability of Amorphous State in Liquid Dispersion

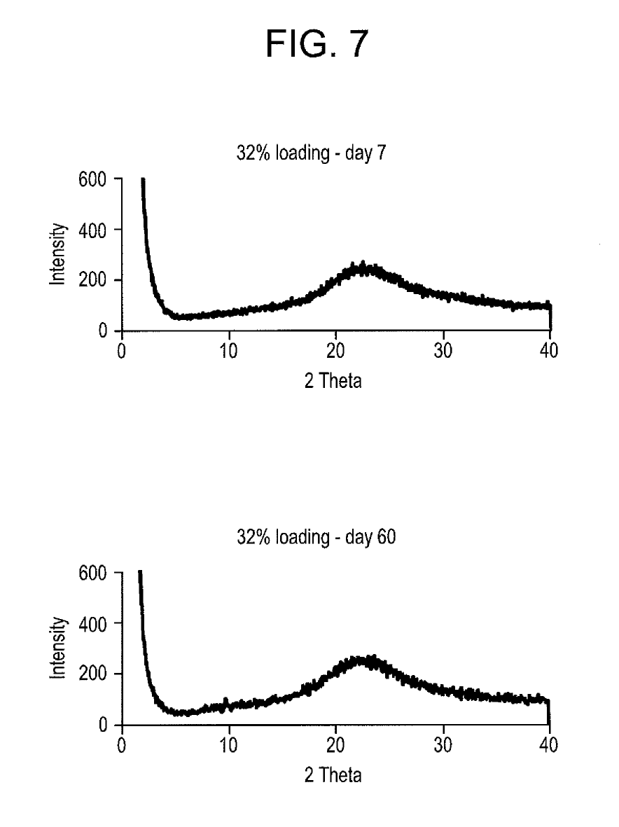

Syloid® SP53D-11920 silica loaded with 32.0% azithromycin (from example 4) incorporated into 5% HPC gel was analyzed by x-ray diffraction as a function of time (x-ray as described in example 4). The x-ray diffraction patterns on day 7 and day 60 showed preserved amorphous state (FIG. 7).

### Example 12

### Pig Ear Penetration Study

### Formulations For the Study:

A weighted amount of 32.0% azithromycin-loaded Syloid® SP53D-11920 silica was incorporated into a 5% HPC gel to get a final concentration of 1% azithromycin loaded in Syloid® SP53D-11920 silica in the gel. 5% raw drug powder with 0.5% TPGS or 5% nanocrystals were incorporated into 5% HPC to get a 5% azithromycin-raw drug powder gel and a 5% azithromycin-nanocrystal gel, respectively. 10% azithromycin-ethanol-solution gel (azithromycin raw drug powder 10%; (94%) ethanol 77.5%; polyacrylate 0.5%; HPC 5%; Miglyol® 812 7%) was selected as a comparison which demonstrated effectiveness in a similar composition in clinical studies (Knauer, J. et al., J. Antimicrob. Chemother. 66 (12), 2814-2822, 2011). To summarize: The Syloid® silica formulation contained 1% azithromycin, the raw drug powder and nanocrystal gel formulations each 5%, and the clinical formulation 10% drug.

Then a penetration study via tape stripping was performed in the pig ear skin model as following: 50 mg 1% azithromycin-Syloid® SP53D-11920 silica gel, 100 mg 5% azithromycin-nanocrystal gel, 100 mg 5% azithromycin-raw drug powder gel and 100 mg 10% azithromycin-ethanol-solution gel were applied homogenously onto a skin area of 1.5×1.5 cm². After a penetration time of 20 minutes, an adhesive tape was pressed onto the skin by using a roller and then removed rapidly. One area was taped for 30 times. Afterwards, the drug was quantitatively extracted from the tape strips using 2 ml of acetonitrile as solvent for shaking 3 hours at 120 rpm in an Innova® 4230 shaker. Subsequently the samples were centrifuged (15493×g; 15 minutes) and the supernatant was analyzed by HPLC.

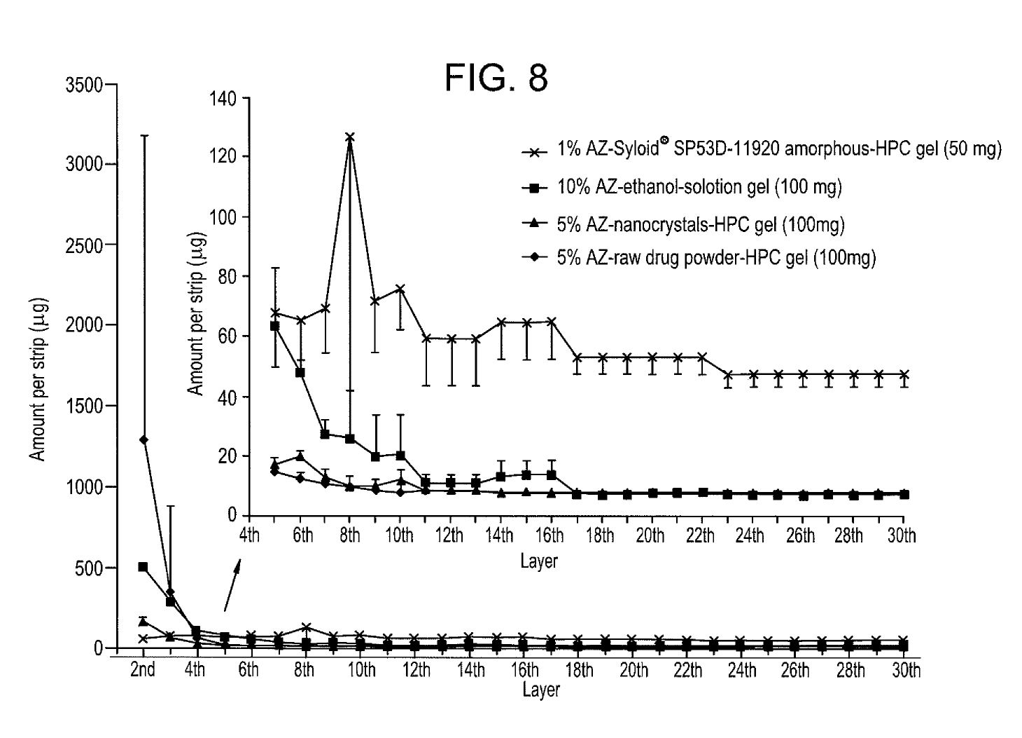

According to FIG. 8, 1% azithromycin-Syloid® SP53D-11920 silica amorphous gel showed higher penetration ability than analogous gel with 5% azithromycin nanocrystals, 5% raw drug powder with TPGS and even higher than the reported 10% azithromycin ethanol gel [1]. The nanocrystals and the gel formulation stayed primarily on the surface of the stratum corneum (2nd and 3rd layer).

### Example 13

### Stability of Syloid® Silica Porous Materials in Dermal Gel Formulations

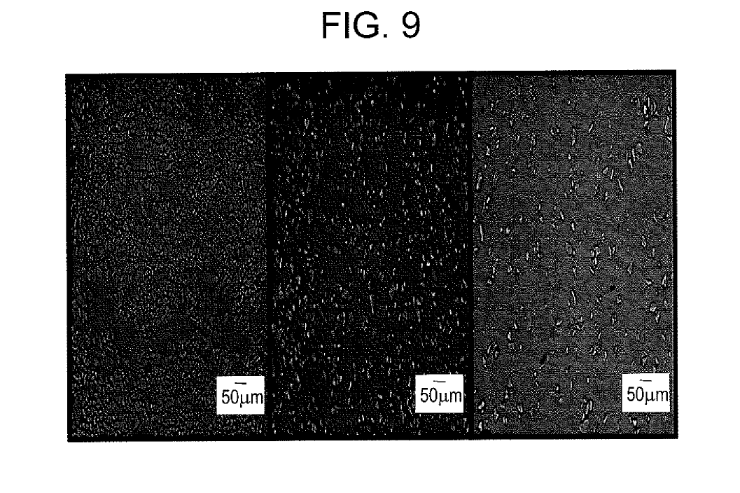

Syloid® SP53D-11920 silica (5% w/w) was dispersed in water and in a 5% hydroxpropylcellulose (HPC, 70 kD) gel and stored for a 4 months at room temperature, and Syloid® SP53D-11920 silica loaded with 30% azithromycin was also dispersed in the HPC gel and stored for 2 months. Light microscopy pictures were taken at 160 fold magnification using an Orthoplan microscope (Leitz, Germany). FIG. 9, left shows some uneven distribution of the Syloid® silica in water with association tendency, but nice stable even distribution and absence of associations in the gels (middle and left).

### Example 14

### Stability of Neusilin® US2 Silica Porous Materials in Dermal Gel Formulations

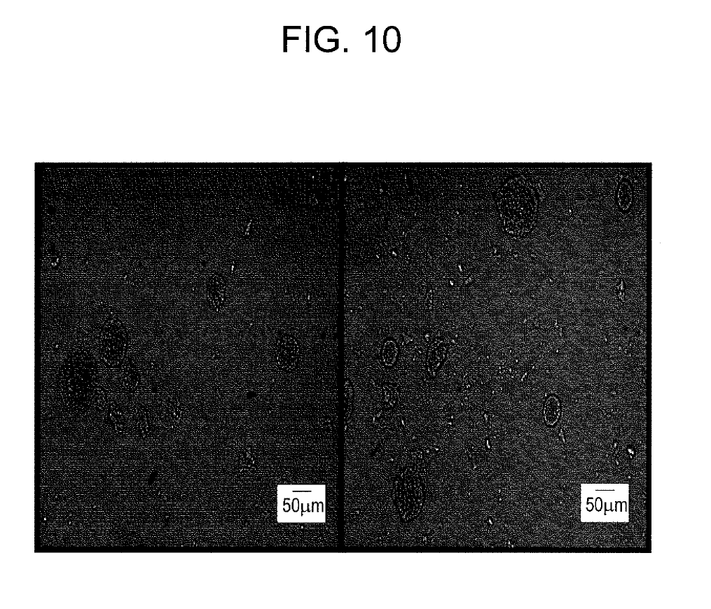

Neusilin® US2 silica (5% w/w) was dispersed in water and in a 5% hydroxpropylcellulose (HPC, 70 kD) gel and stored for a 4 months at room temperature. Light microscopy pictures were taken at 160 fold magnification using an Orthoplan® microscope (Leitz, Germany). FIG. 10 shows even physically stable distribution in water and in the gel.

### Example 15

### Stability of Aeroperl® 300 Silica Porous Materials in Dermal Gel Formulations

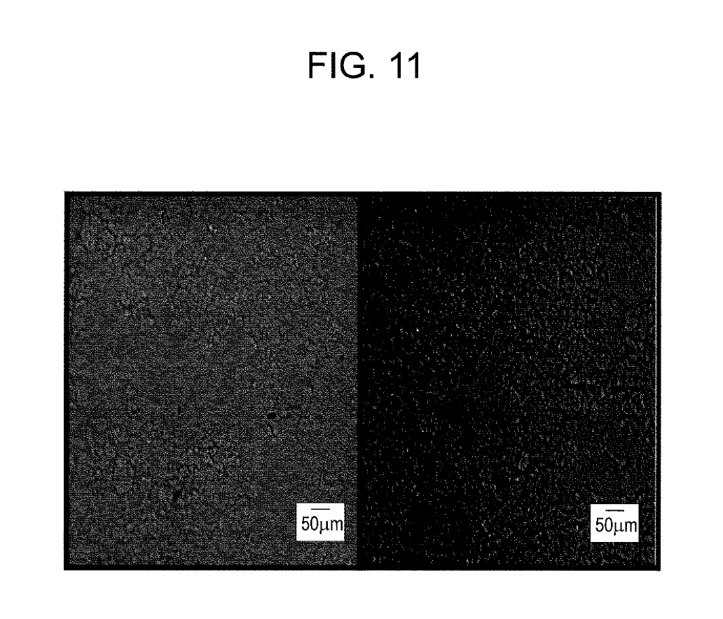

Aeroperl® 300 silica (5 w/w) was dispersed in water and in a 5% hydroxpropylcellulose (HPC, 70 kD) gel and stored for a 4 months at room temperature. Light microscopy pictures were taken at 160 fold magnification using an Orthoplan® microscope (Leitz, Germany). FIG. 11 shows uneven distribution in water with clear association tendency, but even distribution with absence of aggregation in the gel.

### Example 16

### Loading of Aeroperl® 300 Silica With Hesperidin

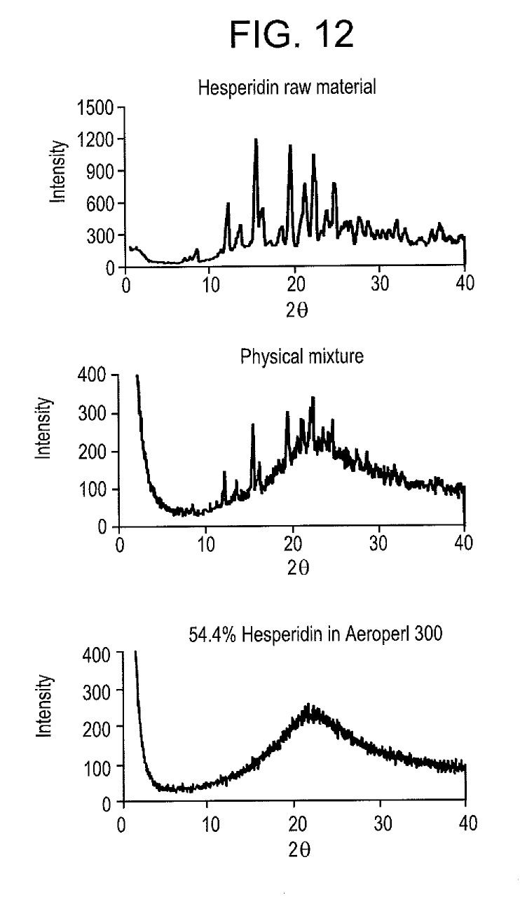

Hesperidin was loaded onto Aeroperl® 300 silica as described in example 4 for loading of Syloid® silica by multiple addition of dissolved hesperidin and subsequent evaporation in a compartment dryer. A loading of 54% could be achieved with hesperidin staying amorphous as analyzed by x-ray dffraction (c.f. example 4) (FIG. 12).

### Example 17

### Loading of Aeroperl® 300 Silica With Hesperidin Under Addition of Surfactant

Aeroperl® 300 silica was loaded with hesperidin dissolved in dimethylsulfoxide (DMSO) under addition of the surfactant Tween 80. Hesperidin solutions with the weight ratio of hesperidin:Tween 80:DMSO=2:1:10, 2:1:15 and 2:1:20 were prepared, respectively. 1 g Aeroperl® 300 silica were added to an ointment bowl and the solutions added in small portions under blending with a mortar. Intermediate drying steps were performed at 80° C. in an compartment dryer, the evaporation controlled by weight loss (weighing in time intervals of 1 hour). With all solutions amorphous products were obtained, loading was performed up to 54%.

### Example 18

### Saturation Solubility of Aeroperl® 300 Silica Loaded With Hesperidin vs. Nanocrystals and Raw Powder

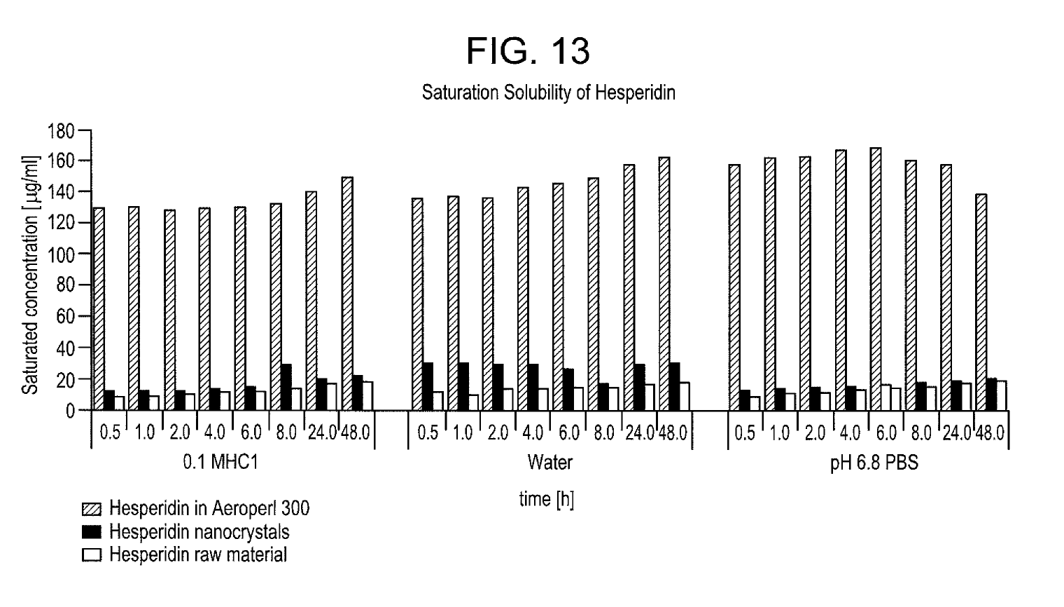

Hesperidin loaded Aeroperl® 300 silica was prepared as described in example 16. Hesperidin nanocrystals were produced applying the combination technology (bead milling using a Miller® PML 2 (Bailer Switzerland) followed by subsequent high pressure homogenization using a Micron LAB® 40 (APV Deutschland, Germany). The saturation solubility was determined in a shaker dispersing hesperidin-loaded Aeroperl® 300 silica, hesperidin nanocrystals and raw powder in water, phosphate buffered saline (PBS) of pH 6.8 and 0.1 M HCl solution at room temperature. FIG. 13 shows an about 5 to 10 fold higher saturation solubility for the hesperidin loaded silica compared to hesperidin nanocrystals with a size of 265 nm.

### Example 19

### Pig Ear Penetration Study for Rutin Loaded on Silica Versus Rutin Nanocrystals

The rutin was loaded onto the silica Syloid® SP53D-11920 silica as described in example 1, but using dimethylsilfoxide as solvent, loading was 32%. For production of the rutin nanocrystals, rutin bulk powder was dispersed in a medium containing 1% (w/w) Tween® 80 and 1% (w/w) Euxyl® PE 9010 with a rutin content of 18% (w/w). The nanosuspension was produced by processing the coarse suspension with 5 passages through the continuous production mode of a wet bead mill PML-2 (Bailer AG, Switzerland) with 0.4-0.6 mm yttrium oxide stabilized zirconium oxide beads (Hosokawa Alpine, Germany) as milling medium at 2,000 rpm rotation speed and 5° C. The batch size was 18 kg. The milled rutin nanosuspension was later diluted to a final rutin concentration of 5%, 2% Tween® 80, 1% Euxyl® PE 9010, 5% glycerol 85% (all weight) and further processed by two cycles of high pressure homogenization (HPH) at 300 bar using a homogenizer Avestin® C50 (Avestin Europe GmbH, Germany). The obtained particle size was 814 nm (zave), determined by photon correlation spectroscopy (PCS) using a Zetasizer® Nano ZS (Malvern Instruments, UK).

A weighted amount of 32.0% rutin-loaded Syloid® SP53D-11920 silica was incorporated into a 5% hydroxypropylcellulose (HPC) gel to get a final concentration of 1% rutin loaded onto Syloid® SP53D-11920 silica in the gel. 5% nanocrystals were incorporated into 5% HPC to get a 5% rutin-nanocrystal gel, respectively. All gels were preserved with 1% Euxyl® PE9010.

Then a penetration study via tape stripping was performed in the pig ear skin model as following: about 50 mg of formulation (1% rutin-Syloid® SP53D-11920 silica gel, 5% rutin-nanocrystal gel) were applied homogenously onto a skin area of 1.5×1.5 cm². After a penetration time of 20 minutes, an adhesive tape was pressed onto the skin by using a roller and then removed rapidly. One area was taped for 19 times. Afterwards, the drug was quantitatively extracted from the tape strips using 2 ml of acetonitrile/DMSO (50:50) as solvent for shaking 3 hours at 120 rpm in an Innova® 4230 shaker. Subsequently these samples were analyzed by HPLC.

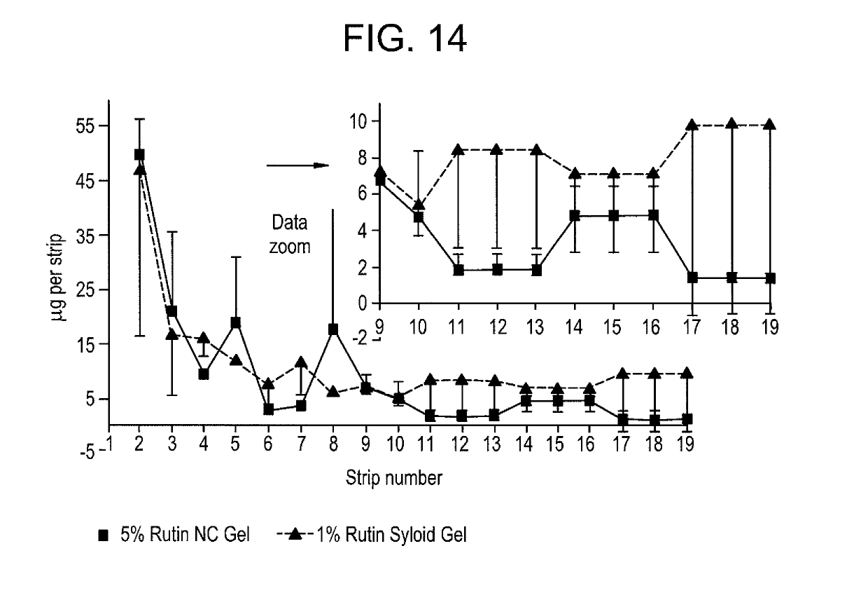

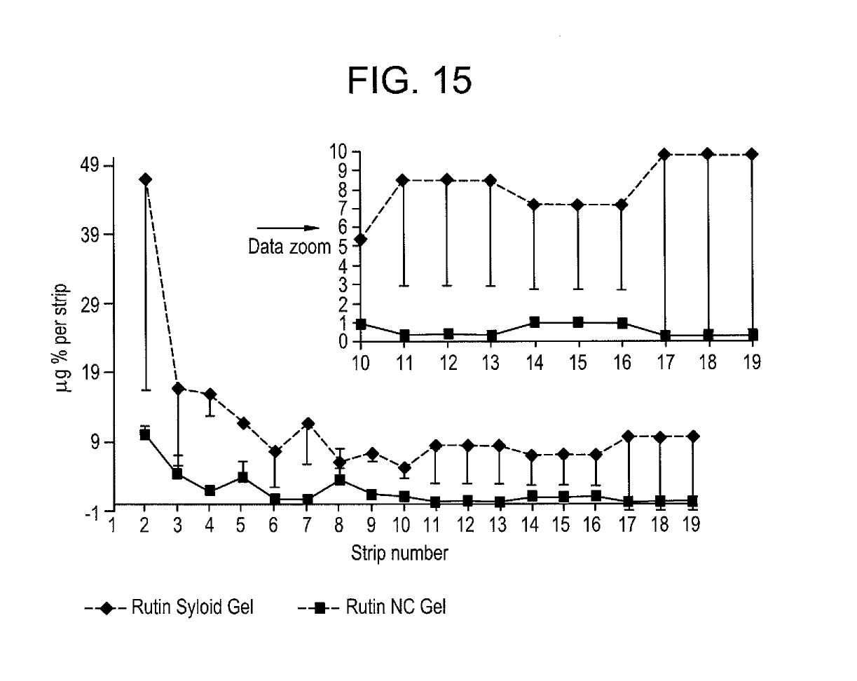

FIG. 14 shows a similar penetration behavior for both rutin nanocrystals and rutin loaded onto Syloid® silca, but in the deeper region the Syloid® silica formulations shows distinctly higher penetrated amounts (μg). However, it has to be considered that the nanocrystal formulation contained 5% rutin but the Syloid® silica formulation only 1%. Strictly speaking a normalization has to be made by dividing the penetrated amount (μg) per tape by the % age of drug in the applied formulation, that means plotting (μg/%) versus the tape number. By doing this, FIG. 15 shows on overall superiority of the Syloid® silica formulation.

### Example 20

### Pig Ear Penetration Study For Hesperidin Loaded on Silica Versus Rutin Nanocrystals

The hesperidin was loaded onto the silica Syloid® SP53D-11920 silica as described in example 1, but using dimethylsilfoxide as solvent, loading was 32%. For production of hesperidin nanosuspensions, the Hesperidin bulk powder was dispersed in a medium containing 1% (wlw) Kolliphor® P 188 and 1% (w/w) Euxyl® PE 9010 with a drug content of 18% (w/w) and processed with the Bühler® PML 2 as described in example 19. The milled hesperidin nanosuspension was later diluted to a final hesperidin concentration of 5%, 1% Kolliphor® P 188, 1% Euxyl® PE 9010, 5% glycerol 85% (all weight) and further processed by one cycle high of pressure homogenization (HPH) at 500 bar using a homogenizer Avestin® C50 (Avestin Europe GmbH, Germany). The obtained particle size was 250 nm (zave), determined by photon correlation spectroscopy (PCS) using a Zetasizer® Nano ZS (Malvern Instruments, UK).

A weighted amount of 32.0% hesperidin-loaded Syloid® SP53D-11920 silica was incorporated into a 5% HPC gel to get a final concentration of 1% hesperidin loaded in Syloid® SP53D-11920 silica in the gel. 5% raw drug powder or 5% nanocrystals were incorporated into 5% HPC to get a 5% hesperidin-raw drug powder gel and a 5% hesperidin-nanocrystal gel, respectively. All gels were preserved with 1% Euxyl® PE9010.

Then a penetration study via tape stripping was performed in the pig ear skin model as following: about 50 mg formulation (1% hesperidin—Syloid® SP53D-11920 silica gel, 5% hesperidin-nanocrystal gel, 5% hesperidin-raw drug powder gel) were applied homogenously onto a skin area of 1.5×1.5 cm², and the study performed as describe in example 19. One area was taped for 30 times.

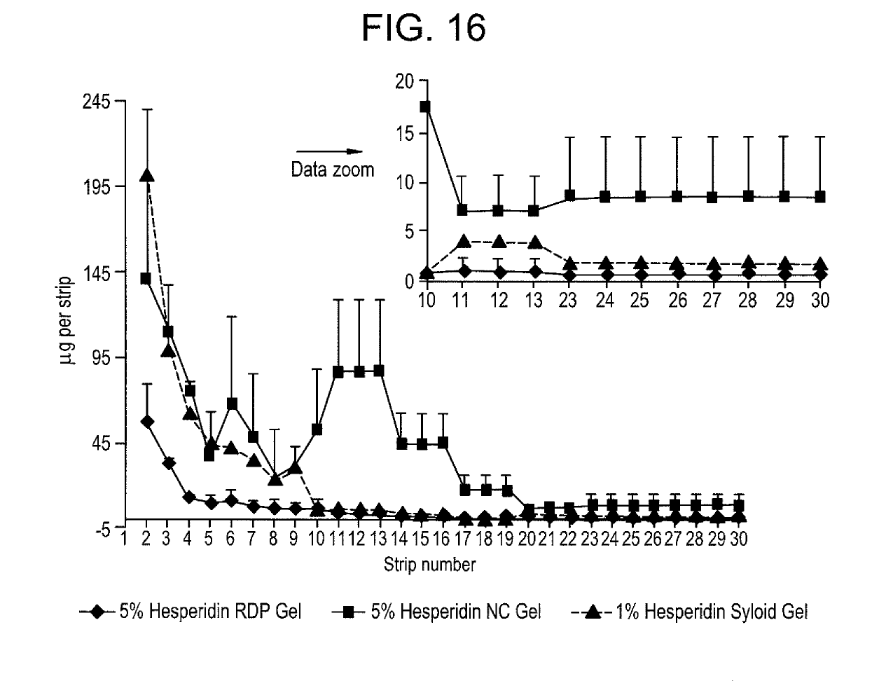

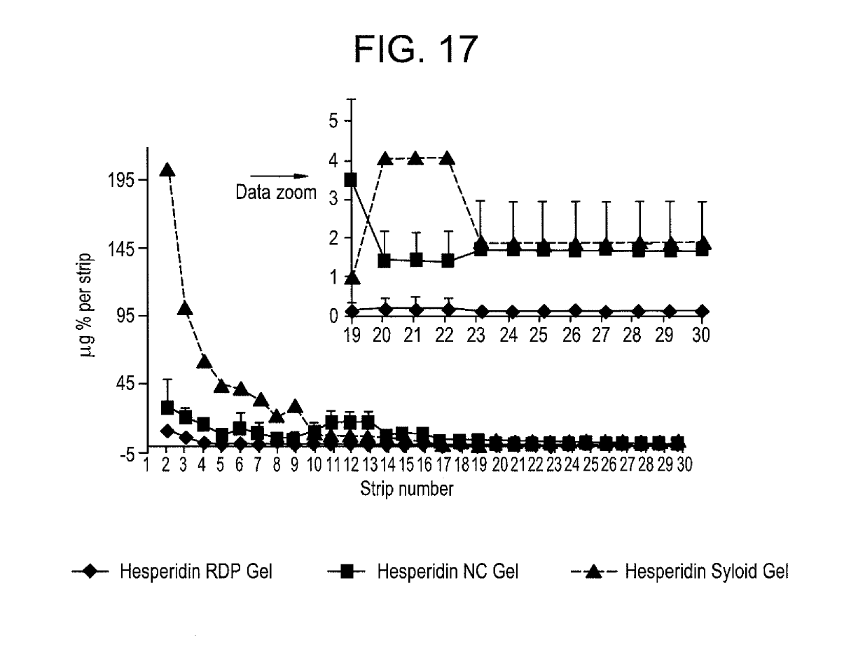

FIG. 16 shows a very low penetration for the raw drug powder, and a similar penetration behavior for both hesperidin nanocrystals and hesperidin loaded onto Syloid® silica until tape 9. In the deeper regions tape 10-20 the nanocrystal formulations is clearly superior in absolute values, below tape 20 slightly superior. However, it has to be considered that the nanocrystal formulation contained 5% hesperidin but the Syloid® silica formulation only 1%. Normalization by dividing the penetrated amount (μg) per tape by the % age of drug in the applied formulation, that means plotting (μg/%) versus the tape number shows a different picture. Related to the concentration applied, the hesperidin Syloid® silica formulation is superior (FIG. 17).

### Example 21

### Pig Ear Penetration Study Amorphous Cyclosporine Particles Versus Cyclosporine Loaded on Silica

The cyclosporine was loaded onto the silica Syloid® SP53D-11920 silica as described in example to achieve Syloid® SP53D-11920 silica with cyclosporine—loading 32% (w/w). First the drug cyclosporine raw powder was dissolved in ethanol (96%) in a ratio of 1:4 by weight to get cyclosporine ethanol solution. Then 32.0% loading Syloid® SP53D-11920 silica was achieved by 3 steps. In the first step, 2.5 g Syloid® SP53D-11920 silica was loaded with 0.5 g drug by addition of 2.5 g solution under stirring using an ointment bowl and pestle. To ensure that the drug solution was absorbed by the silica immediately and homogenously, the cyclosporine solution was sprayed manually by a spraying nozzle screwed onto a glass bottle. Subsequently the ethanol was evaporated at 40° C. in a compartment dryer. The complete evaporation was controlled via determining the weight loss. In the second step, 2.25 g of the obtained silica was loaded with 0.4 g drug by spraying of 2 g solution using the same method. In the third step, 2.025 g of this silica was loaded with 0.186 g drug by spraying of 0.93 g solution.

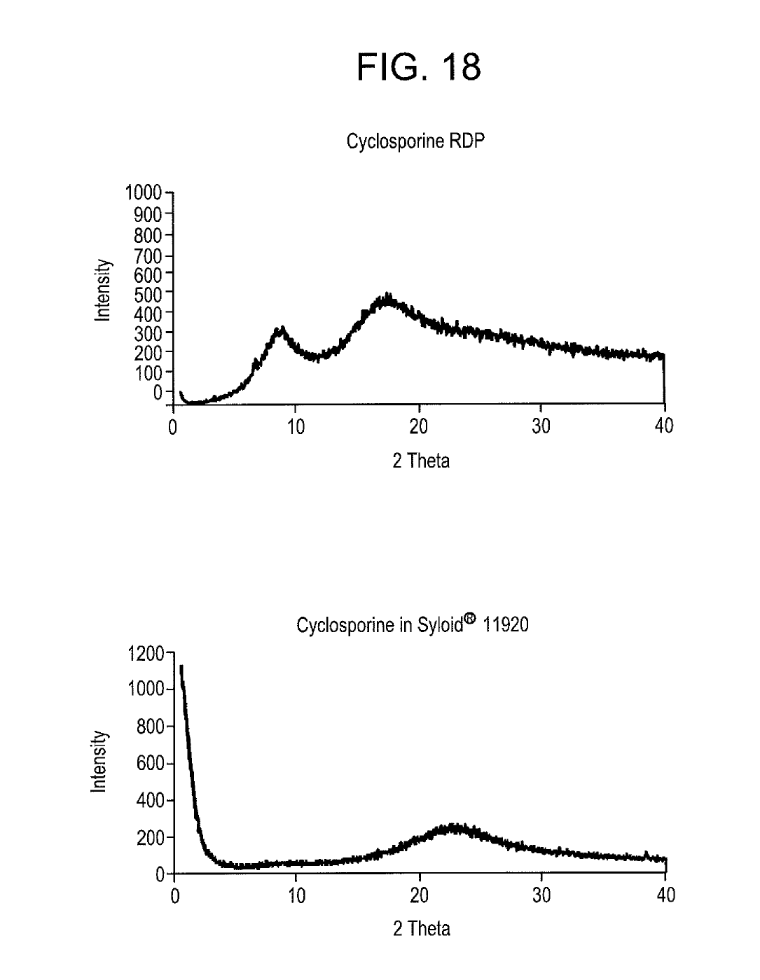

The raw drug powder and the cyclosporine loaded Syloid® silica were analyzed by x-ray diffraction as described in example 4, which showed the amorphous state of both formulations (FIG. 18).

A weighted amount of cyclosporine-loaded Syloid® SP53D-11920 silica was incorporated into a 5% hydroxypropylcellulose (HPC) gel to get a final concentration of 1% cyclosporine loaded in Syloid® SP53D-11920 silica in the gel. 5% raw drug powder was incorporated into 5% HPC to get a 5% cyclosporine-raw drug powder gel. All gels were non-preserved. Then a penetration study via tape stripping was performed in the pig ear skin model as described in example 20, one area was tape stripped 30 times.

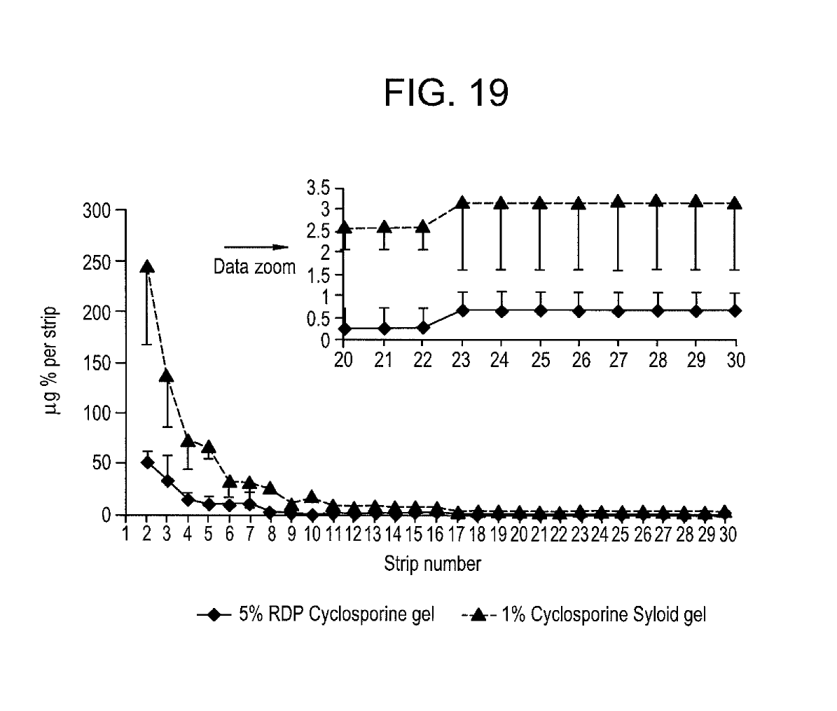

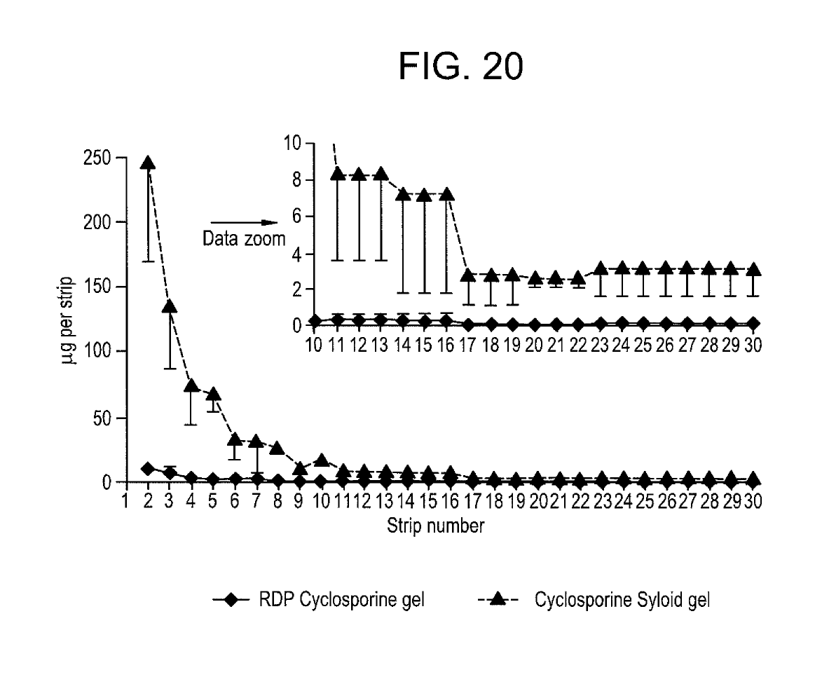

FIG. 19 shows a clearly superior penetration of cyclosporine from the Syloid® silica formulation—despite that the cyclosporine powder was in the amorphous state. Penetration is very pronounced superior in the deeper layers (tape strips, 20-30). Obviously an amorphous state loaded onto porous materials leads to a better skin penetration. This is even more obvious when looking at the normalized plot (FIG. 20). Identical to examples 19 and 20 normalization was performed by dividing the penetrated amount (μg) per tape by the % age of drug in the applied formulation. Plotting (μg/%) versus the tape number shows even better the superiority of the Syloid® silica formulation, with up to about 25 fold higher amounts in the strips.

1. A composition comprising one or more porous particulate materials which are loaded with one or more biological active in the amorphous form, either inside the pores, on the surface, or both inside pores and on the surface, wherein the porous particulate materials have an average pore size of about 2 to about 250 nm.

2. The composition according to claim 1 in which the porous particulate material comprises at least one inorganic material.

3. The composition according to claim 2 in which the porous particulate material comprise a porous inorganic oxide.

4. The composition according to claim 1 in which the porous particulate material comprises at least one organic material selected from the group consisting natural and synthetic polymers (c.g. from lactic and glycolic acid).

5. The composition according to claim 1 in which the biological active is in a substantially amorphous form.

6. The composition according to claim 1 in which the biological active is in a partially amorphous form.

7. The composition according to claim 6 in which the biological active has a crystallinity of less than 50% as determined by x-ray diffraction or by differential scanning calorimetry (DSC).

8. The composition according to claim 1 in which the porous particulate material has a pore volume of about 0.1 cm³/g or greater.

9. The composition according to claim 1, in which the porous particulate material has an average pore diameter of about 2 nm to about 200 nm.

10. The composition according to claim 9 in which the porous particulate material has an average pore diameter of from about 50 nm to about 250 nm.

11. The composition according to claim 1 in which the porous particulate material has a BET surface area from about 10 m²/g to about 1000 m²/g.

12. The composition according to claim 1 in which the porous particulate material has a average particle size of from about 0.1 μm to about 1,000 μm.

13. The composition according to claim 12 in which the average particle size of the porous particulate material is less than 125 μm.

14. The composition according to claim 12 in which the average particle size of the porous particulate material is 125 μm or greater.

15. The composition according to claims 1, in which the porous particular material comprises silica particles.

16. The composition according to claim 15, in which the silica particles further comprises metal ions.

17. The composition according to claim 16 wherein the metal ions are selected from the group comprising alkali metals, earth alkali metals, transition metals, post transition metals, metalloids and combinations thereof.

18. The composition according to claim 16 in which the concentration of metal ions is up to about 80 wt % (on an oxide basis) of the total silica particles.

19. The composition according to claim 18 in which the concentration of metal ions is about 50 wt % or less (on an oxide basis) of the total silica particles.

20. A composition according to claim 15 in which the silica particles are selected from the group comprising amorphous silicon dioxide, fumed silica, precipitated silica, colloidal silica, bimodal silica, ordered pore silica, non-ordered pore silica and combinations thereof.

21. The composition according to claim 1 in which the biological active is an active selected from the group comprising pharmaceutical, a cosmetic, cosmeceutical or a combination thereof.

22. The composition according to claim 21 in which the biological active is a pharmaceutical active selected from the group comprising nonsteroidal anti-inflammatory drugs, reverse-transcriptase inhibitors, antibiotics, peptides, corticosteroids and mineral ocorticoids, aromatase inhibitors, antifungal drugs and a combination thereof.

23. The composition according to claim 21 wherein the biological active is an active selected from the group comprising quinones, flavanoids, carotinoids, xanthyphylls, stilbenoids and dihydro-stilbenoids and a combination thereof.

24. The composition according to claims 1 in which the composition further comprises excipients selected from the group comprising surfactant/stabilizers, polymers, gelling agents, water wettability reducing hydrophobic compounds, and a combination thereof.

25. The composition according to claim 24, in which the surfactant/stabilizer is selected from the group comprising anionic surfactants, cationic surfactants, nonionic surfactants/stabilizers, glycerol alkyl esters, sorbitan alkyl esters, cocamide monoethanolamine, dodecyldimethylamine oxide, block copolymers of polyethylene glycol and polypropylene glycol, polyethoxylated tallow amine, alkylphenol ethoxylates, alkyl polyglycoside, tocopheryl polyethylene glycol 1000 succinate, polysorbates, zwitterionic surfactants and combinations thereof.

26. The composition according to claim 24 in which the polymer is selected from the group comprising copolymers of polyoxypropylene and polyoxyethylene, polyethers, polyvinylesters, polysaccharides, cellulose derivatives, polyacrylic acids, polyvinyl alcohols and combinations thereof.

27. The composition according to claim 24, in which the gelling agent is selected from the group comprising polyoxyethylene-propylene blockcopolymers, polysaccharides, cellulose derivatives, starch and starch derivates, alginates, polyacrylic acids, silicas, gelatins, bentonites and combinations thereof.

28. The composition according to claim 24, in which the water wettability reducing hydrophobic compound is a lipid or a natural or synthetic hydrocarbon.

29. The composition according to claim 1 in which the composition further comprises nanoparticles.

30. The composition according to claim 29 in which the nanoparticles is selected from the group comprising nanocrystals, solid lipid nanoparticles, nanostructured lipid carriers, liposomes or a combination thereof.

31. A dermal or topical composition for use on the human or animal skin or mucosa to deliver a biological active, comprising the composition of claims 1.

32. The dermal or topical composition according to claim 31 wherein the biological active is dispersed in a liquid media.

33. The dermal or topical composition according to claim 32 in which the liquid media comprise water, an aqueous solution, an oil, a hydrocarbon, an organic solvent or a combination thereof.

34. The dermal or topical composition according to claims 31 in which the composition has an outer continuous phase.

35. The dermal or topical composition according to claim 34 in which the outer continuous phase comprises an aqueous or non-aqueous gel system.

36. The dermal or topical composition according to claim 34, in which the outer continuous phase comprise an oil-in-water cream or a water-in-oil cream.

37. The composition according to claim 34, in which the outer continuous phase comprises a semi-liquid phase, a highly viscous phase or a solid phase.

38. The composition according to claim 37 in which the semi-liquid phase is selected from viscous oils, Vaseline, or petroleum jelly.

39. The composition according to claim 37 in which the highly viscous phases is a semi-solid phase or solid phase.

40. The composition according to claim 37 wherein the solid phase is a polymer matrix of a dermal or transdermal patch or polymers based on acrylic esters such as 2-EHA (2-Ethylhexyl acrylate) and ethyl acrylate.

41. A method of using of the composition of claim 1 for delivering at least one biological active into the skin or mucosa of a human or animal.

42. A method of enhancing the dermal or topical delivery of a biological active comprising administering a biologically effective amount of a composition according to claim 1 to the skin or mucosa of an human or animal.

43. A dermal or topical composition for use in humans or animal, which composition comprises a composition in accordance claim 1.

44. The composition according to the claim 43 wherein the composition is applied to hair follicles of a human or an animal for delivery of at least one biological active in the skin.

45. The composition according to claim 44 wherein the porous particles possess a size less than 20 μm.

46. The composition according to claim 45 where in the porous particles are dispersed in a low viscosity formulation.

47. The composition according to the claim 43 wherein the composition is applied to the oral cavity or pharynx of a human or an animal for oromuscosal delivery of at least one biological active.

48. The composition according to claim 47, wherein the porous particles are incorporated into and/or applied in the form of a suspension, a gel, a cream, an ointment, a liquid spray, a powder spray, mucosal films or patches, lozenges, oral disintegrating tablets (ODT), or chewing gums.

49. The composition according to claim 43 wherein the composition is applied to the nasal cavity of a human or an animal for delivery of at least one biological active.

50. The composition according to claim 49, wherein the particle size of the porous particles is less than 50 μm.

51. The composition according to claim 49 wherein the porous particles are incorporated into and/or applied in the form of a suspension, nasal drops, a gel, a cream, an ointment, a liquid spray, a powder spray, or in a nasal tampon.

52. The composition according to claim 51 wherein the nasal cavity is the upper nasal cavity

53. The composition according to claim 52 wherein delivery of the biological active is intended for the brain of a human or animal.

54. The composition according to the claim 43 wherein the composition is applied to the surface of the eye of a human or an animal for ocular delivery of at least one biological active.

55. The composition according to claim 54 wherein the porous particles have a particle size of less than 10 μm.

56. The composition according to claim 54 wherein the composition is incorporated into and/or applied in the form of a liquid suspension, gel, self-gelling gel, cream, an ointment, eye contact lenses, or injectable implants from which the actives diffuse into the surface of the eye.

57. A method comprising using a dermal or topical composition in the hair follicles of a human or animal for delivering of at least one biological active into the skin, which composition comprises a composition in accordance claim 44.

58. A method comprising using a dermal or topical composition in the nasal cavity of a human or animal for delivering at least one biological active into the mucosa of the nasal cavity, which composition comprises a composition in accordance with claim 49.

59. A method comprising using a dermal or topical composition wherein the composition is used in the upper nasal cavity for delivery of at least one biological active to the brain of a human or animal, which compositions comprises a composition in accordance with claim 52.

60. A method comprising using a dermal or topical composition for ocular delivery of at least one biological active to the surface of the eye of a human or an animal, which composition comprises a composition in accordance with claim 54.

61. A method comprising using a dermal or topical composition for oromuscosal delivery of at least one biological active to the oral cavity of a human or an animal, which composition comprises a composition in accordance with claim 47.
